(19) United States
(12) Patent Application Publication
(10) Pub. No.: US 2019/0175026 A1
(43) Pub. Date: Jun. 13, 2019

(54) CARDIAC AND SLEEP MONITORING

(71) Applicant: INSPIRE MEDICAL SYSTEMS, INC., Maple Grove, MN (US)

(72) Inventors: Kevin Verzal, Maple Grove, MN (US); Kent Lee, Maple Grove, MN (US); John Rondoni, Maple Grove, MN (US); Dave Dieken, Maple Grove, MN (US)

(73) Assignee: INSPIRE MEDICAL SYSTEMS, INC., Maple Grove, MN (US)

(21) Appl. No.: 15/771,298

(22) PCT Filed: Nov. 11, 2016
(86) PCT No.: PCT/US2016/061672

§ 371 Date: Nov. 2, 2018

Related U.S. Application Data
(60) Provisional application No. 62/253,803, filed on Nov. 11, 2015.

(51) Int. Cl. A61B 5/0205; A61B 5/00; A61B 5/024; A61N 1/372

(52) U.S. Cl. CPC A61B 5/0205; A61B 5/4818; A61B 5/046; A61B 5/4836; A61N 1/37254; A61B 5/02405

(57) ABSTRACT

An apparatus to monitor at least one sleep parameter and/or at least one cardiac parameter.

### CROSS-REFERENCE TO RELATED APPLICATIONS

This application is a National Stage Application that claims priority to PCT Application No. PCT/US2016/061672, entitled “CARDIAC AND SLEEP MONITORING,” having a filing date of Nov. 11, 2016 which is a Non-Provisional application which claims the benefit of Provisional U.S. Patent Application Ser. No. 62/253,803, entitled “CARDIAC MONITORING IN ASSOCIATION WITH SLEEP DISORDERED BREATHING-RELATED DEVICE,” having a filing date of Nov. 11, 2015, both of which are incorporated herein by reference.

### BACKGROUND

Treating sleep disordered breathing has led to improved sleep quality for some patients.

### BRIEF DESCRIPTION OF THE DRAWINGS

FIG. 1 is a block diagram schematically representing an arrangement including a monitoring resource for cardiac-related information, according to one example of the present disclosure.

FIG. 2A is a block diagram schematically representing a cardiac disorder parameter, according to one example of the present disclosure.

FIG. 2B is a block diagram schematically representing a cardiac health parameter, according to one example of the present disclosure.

FIG. 3A is a block diagram schematically representing an arrangement including a monitoring resource, according to one example of the present disclosure.

FIG. 3B is a block diagram schematically representing access tools, according to one example of the present disclosure.

FIG. 3C is a block diagram schematically representing a user interface, according to one example of the present disclosure.

FIG. 4A is a diagram schematically representing one instance of a clinician user interface, according to one example of the present disclosure.

FIG. 4B is a table schematically representing aspects of an import function associated with a clinician user interface, according to one example of the present disclosure.

FIG. 4C is a table schematically representing aspects of a filter function associated with a clinician user interface, according to one example of the present disclosure.

FIG. 4D is a table schematically representing a correlation coefficient array for a plurality of sleep parameters and a plurality of cardiac parameters, according to one example of the present disclosure.

FIG. 4E is a table schematically representing a correlation coefficient relationship for one sleep parameter and one cardiac parameter, according to one example of the present disclosure.

FIG. 4F is a diagram including a pair of graphs schematically representing the information in the table of FIG. 4E.

FIG. 5A is a table schematically representing information regarding a cardiac parameter and a sleep parameter in one instance of a patient user interface, according to one example of the present disclosure.

FIG. 5B is a graph schematically representing information regarding a cardiac parameter and a sleep parameter in one instance of a patient user interface, according to one example of the present disclosure.

FIG. 6A is a block diagram schematically representing stimulation circuitry, according to one example of the present disclosure.

FIG. 6B is a block diagram schematically representing upper-airway-related body tissue, according to one example of the present disclosure.

FIG. 6C is a block diagram schematically representing a non-cardiac pulse generator, according to one example of the present disclosure.

FIG. 7 is a block diagram schematically representing stimulation therapy components, according to one example of the present disclosure.

FIG. 8 is a block diagram schematically representing therapy modalities, according to one example of the present disclosure.

FIG. 9 is a block diagram schematically representing types of information associated with a therapy device, according to one example of the present disclosure.

FIG. 10A is a block diagram schematically representing a stimulation therapy device including a sensor, according to one example of the present disclosure.

FIG. 10B is a block diagram schematically representing a stimulation therapy device separate from a sensor, according to one example of the present disclosure.

FIG. 11 is a block diagram schematically representing sensors, according to one example of the present disclosure.

FIG. 12 is a block diagram schematically representing sensor types, according to one example of the present disclosure.

FIG. 13A is a diagram schematically representing some aspects of accelerometer sensing in association with some aspects of sleep quality, according to one example of the present disclosure.

FIG. 13B is a diagram schematically representing some aspects of accelerometer sensing in association with some aspects of sleep quality, according to one example of the present disclosure.

FIG. 13C is a diagram schematically representing some aspects of acoustic sensing of cardiac information and respiratory information, according to one example of the present disclosure.

FIG. 13D is a diagram schematically representing a Wiggers Diagram, according to one example of the present disclosure.

FIG. 13E is a diagram schematically representing non-contact sensing of respiratory information, according to one example of the present disclosure.

FIG. 13F is a diagram schematically representing derivation of respiratory information from a cardiac waveform, according to one example of the present disclosure.

FIG. 13G is a diagram schematically representing a juxtaposition of cardiac timing information and respiratory information, according to one example of the present disclosure.

FIG. 13H is a diagram schematically representing a juxtaposition of respiratory information, cardiac information, and sleep information, according to one example of the present disclosure.

FIG. 13I is a diagram schematically representing an overnight patient report including cardiac information, respiratory information, and sleep information, and, according to one example of the present disclosure.

FIG. 14A is a block diagram schematically representing an array of sensor modalities, according to one example of the present disclosure.

FIG. 14B is a block diagram schematically representing a sensor profile manager associated with a therapy device, according to one example of the present disclosure.

FIG. 15A is a block diagram schematically representing a cardiac condition array, according to one example of the present disclosure.

FIG. 15B is a block diagram schematically representing a cardiac condition determination engine, according to one example of the present disclosure.

FIG. 15C is a block diagram schematically representing a determination engine, according to one example of the present disclosure.

FIG. 16A is a block diagram schematically representing a therapy system including cardiac monitoring, according to one example of the present disclosure.

FIG. 16B is a diagram schematically representing a therapy system as deployed on a patient, according to one example of the present disclosure.

FIG. 16C is a block diagram schematically representing at least some components of a pulse generator, according to one example of the present disclosure.

FIG. 17A is a block diagram schematically representing a monitoring resource including a sensor, according to one example of the present disclosure.

FIG. 17B is a block diagram schematically representing a monitoring resource, according to one example of the present disclosure.

FIG. 18A is a block diagram schematically representing a manager, according to one example of the present disclosure.

FIG. 18B is a table listing at least some sleep quality parameters, at least some cardiac parameters, and other parameters, according to one example of the present disclosure.

FIG. 18C is a diagram of a correlation graphing tool, according to one example of the present disclosure.

FIG. 19 is a block diagram schematically representing a therapy device, according to one example of the present disclosure.

FIG. 20 is a block diagram schematically representing a wireless communication link, according to one example of the present disclosure.

FIG. 21 is a block diagram schematically representing a sensor, according to one example of the present disclosure.

FIG. 22 is a block diagram schematically representing an evaluation engine, according to one example of the present disclosure.

FIG. 23 is a block diagram schematically representing a control portion, according to one example of the present disclosure.

FIG. 24A is block diagram schematically representing instructions for cardiac monitoring, according to one example of the present disclosure.

FIG. 24B is block diagram schematically representing instructions for cardiac monitoring, according to one example of the present disclosure.

FIG. 25 is a flow diagram schematically representing instructions for cardiac monitoring, according to one example of the present disclosure.

FIGS. 26-28 are block diagrams schematically representing instructions for sleep parameter monitoring and/or cardiac parameter monitoring, according to some examples of the present disclosure.

FIG. 29 is a block diagram schematically representing instructions for displaying information, according to one example of the present disclosure.

### DETAILED DESCRIPTION

In the following Detailed Description, reference is made to the accompanying drawings which form a part hereof, and in which is shown by way of illustration specific examples of the present disclosure which may be practiced. In this regard, directional terminology, such as “top,” “bottom,” “front,” “back,” “leading,” “trailing,” etc., is used with reference to the orientation of the Figure(s) being described. Because components of at least some examples of the present disclosure can be positioned in a number of different orientations, the directional terminology is used for purposes of illustration and is in no way limiting. It is to be understood that other examples may be utilized and structural or logical changes may be made without departing from the scope of the present disclosure. The following detailed description, therefore, is not to be taken in a limiting sense.

At least some examples of the present disclosure are directed to cardiac monitoring and/or sleep monitoring. In at least some examples of the present disclosure, cardiac monitoring may be employed in association with a therapy for sleep disordered breathing. In some instances, such cardiac monitoring may help demonstrate long term efficacy of sleep disordered breathing therapy in improving cardiac health or in slowing down progression of negative cardiac conditions (e.g. cardiac disorders). In some instances, such cardiac monitoring may help identify negative cardiac conditions which are not alleviated despite efficacious sleep disordered breathing therapy, and thereby facilitate the diagnosis and treatment of such cardiac conditions.

In some examples, such cardiac monitoring is performed via obtaining physiologic-related information. In some examples, the cardiac monitoring is performed in association with the sleep disordered breathing treatment and then deriving or extracting cardiac information from the physiologic-related information. In some examples, this physiologic-related information may include at least respiratory information. Accordingly, in some examples, cardiac monitoring is performed via at least some of the components associated with a therapy device for treating sleep disordered breathing.

However, in some examples, such cardiac monitoring is performed via obtaining cardiac information independent of obtaining other physiologic-related information. Accordingly, in some examples, cardiac monitoring is performed via devices or components separate from, and independent of, a therapy device for treating sleep disordered breathing.

For instance, in some examples such cardiac monitoring may be performed via a monitoring resource, whether or not a therapy device is involved. In at least some examples, a monitoring resource can take a variety of forms. In some examples, at least a portion of the monitoring resource is located within an implantable element the patient and/or within the presence of the patient, such as within components external to, but near, the patient. In some examples, at least a portion of the monitoring resource is located remotely from the patient, such as in an implementation via a server, other computing device, which may be located in the cloud (e.g. web-based computing resource) or in a monitoring facility (e.g. clinic, device manufacturer facility, hospital, etc.).

Whether or not near the patient, in some examples at least a portion of the monitoring resource may be located in and/or accessible via a dedicated mobile device (e.g. patient or clinician remote control) or a non-dedicated mobile device (e.g. smart phone, tablet, etc.). In some such examples, the monitoring resource may be implemented via an app (e.g. mobile application), widget, and/or other computing/communication resource operable via such mobile devices. In some examples, regardless of location at least a portion of the monitoring resource may be implemented via a stationary device, e.g. a workstation.

For instance, in some examples a monitoring resource monitors information without displaying the monitored information. However, in some examples, at least some of the monitored information is displayable. Accordingly, in some examples, monitoring information does not necessitate displaying such information.

In some examples, regardless of location, the monitoring resource may be at least partially implemented via a user interface through which at least some features, functions, and attributes of the monitoring resource may be displayed, accessed, engaged, etc. In some such examples, the user interface is accessible as a clinician user interface, a patient interface, etc. whether available via a web interface, mobile app, program (e.g. desktop, notebook computer), etc.

In some examples, monitoring via the monitoring resource comprises observing a parameter (e.g. sleep, cardiac, etc.) over a period of time. In some instances, the monitoring of one or more parameters over a period of time may sometimes be referred to as tracking the parameter at least in the sense the parameters are observed over time.

In some examples, the monitoring comprises receiving information regarding the parameter without performing a measurement. In some such examples, the information may be received from an external source, such as environmental information, patient history, etc. However, in some examples, the monitoring comprises monitoring the parameter via sensing information via at least one sensor. In some instances, the sensing may include or be associated with measuring.

In some examples, the monitoring comprises determining further information or drawing a conclusion, such as whether a particular parameter may be associated with or at least partially define a condition. For instance, upon monitoring a particular cardiac parameter, the monitoring may determine that a cardiac condition (e.g. atrial fibrillation) is exhibited. It will be understood that in at least some examples, the cardiac condition may be considered part of and/or encompassed by an associated cardiac parameter. Similarly, upon monitoring a particular sleep parameter, the monitoring may determine that a sleep condition (e.g. obstructive sleep apnea) is exhibited. In some examples, such determining may include determining correlations, trends between among different monitored parameters, determining to provide a notification to a patient or clinician, etc.

Accordingly, it will be understood that in at least some examples, the term “monitoring” and the term “monitoring resource” may broadly encompass determining, observing, receiving, sensing, measuring, tracking, displaying, etc. a parameter relating to at least sleep parameters and/or cardiac parameters. However, it will be understood that the various different features, functions, attributes, etc. associated with the term “monitoring” and/or “monitoring resource” may be distinct from each other, while existing in a complementary manner in at least some examples.

In some examples, “monitoring” and/or “the monitoring resource” are associated with a monitoring period. However, in some examples, “monitoring” and/or “a monitoring resource” are not associated with a particular monitoring period.

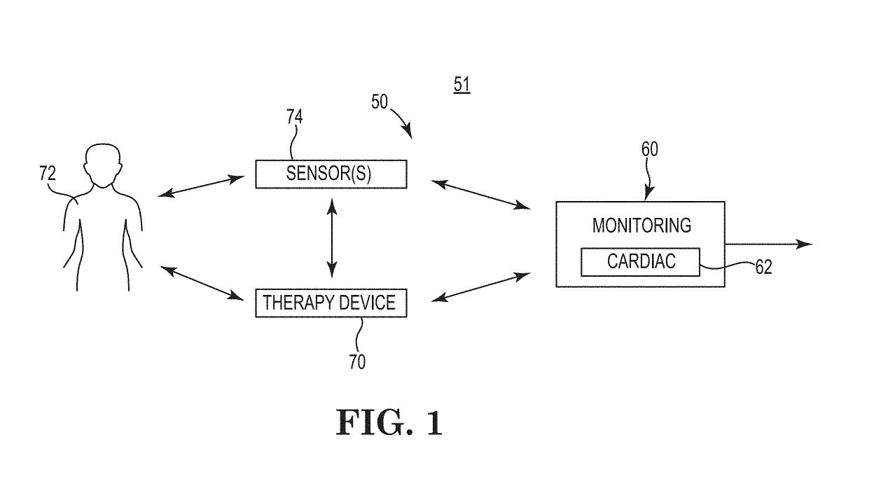

Moreover, at least some of these features, functions, and attributes of a monitoring resource, and/or additional features, functions, and attributes of a monitoring resource, are further defined in the context of at least some examples of the present disclosure in association with FIGS. 1-29.

These examples, and additional examples, are described in more detail in association with at least FIGS. 1-29.

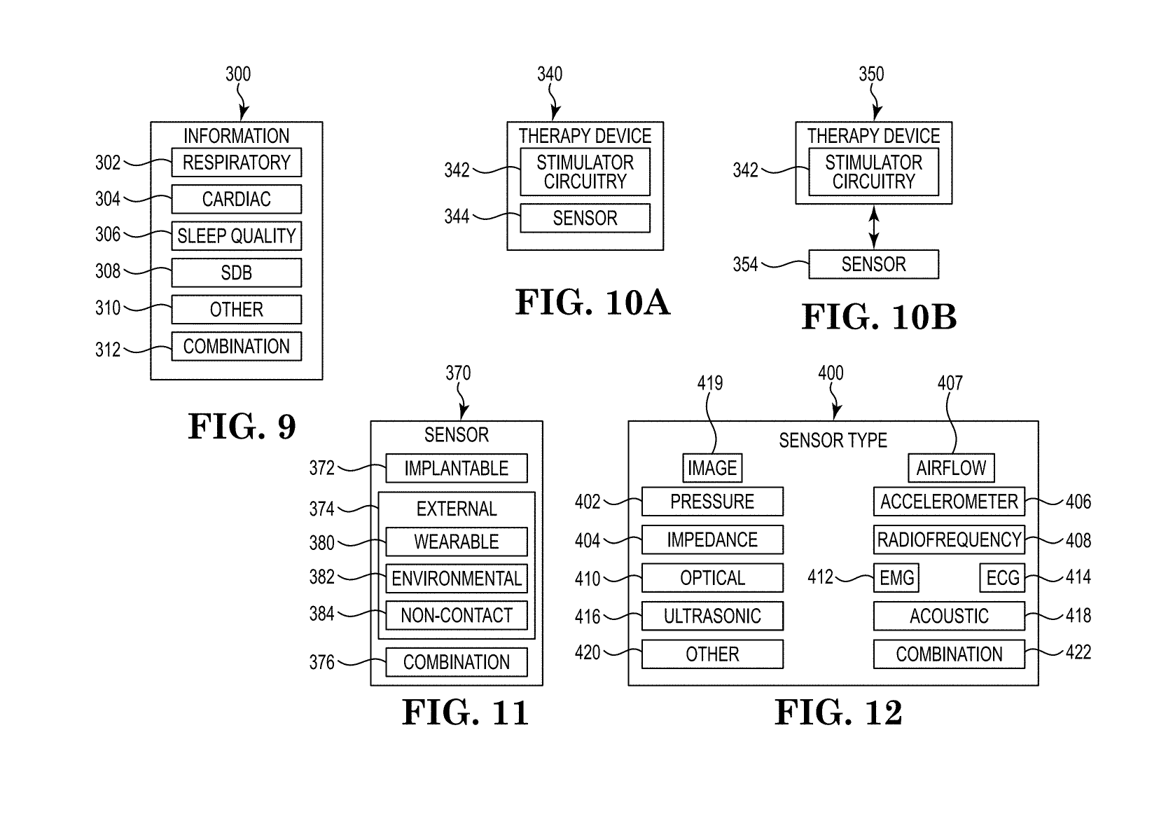

FIG. 1 is a block diagram 51 schematically representing a monitoring resource 60 in an arrangement 50, according to one example of the present disclosure. As shown in FIG. 1, in some examples arrangement 50 comprises a monitoring resource 60 to monitor and/or evaluate information regarding a patient 72. In some examples, the information may comprise physiologic-related information and/or other information (e.g. environmental information) indicative of cardiac-related information. In some examples, the information also may comprise information regarding sleep quality, which may include information regarding sleep disordered breathing (SDB) behavior. In some examples, SDB behavior comprises obstructive sleep apneic behavior. In some examples, the information may comprises at least one of the types of information as further described later in association with at least FIG. 9.

In some examples, monitoring resource 60 obtains such information via at least one sensor 74. The sensor(s) 74 may be implantable, external, contact, non-contact, etc. as further described later in association with at least FIGS. 11-12, and may be in wired or wireless communication with monitoring resource 60. In some instances, the sensor(s) 74 may be incorporated into monitoring resource 60.

In some examples, the arrangement 50 may comprise a therapy device 70. In such arrangements, in some examples the monitoring resource 60 may receive information from the therapy device 70 regarding the patient 72 and/or a therapy applied to the patient. In some examples, monitoring resource 60 may communicate information to the therapy device 70, which may be used in some examples to determine therapy parameters. In some examples, monitoring resource 60 may communicate wirelessly with therapy device 70.

In some examples, information from sensor(s) 74 may be received by therapy device 70, which in turn may be communicated to the monitoring resource 60 in some examples.

In some examples, monitoring resource 60 receives patient-related information from external sources other than sensor(s) 74 and/or therapy device 40.

In general terms, the therapy device 70 can take a variety of forms provided that it works toward alleviating sleep disordered breathing (e.g. obstructive sleep apneas) in the patient 72. In some examples, therapy device 70 provides neurostimulation to upper-airway-related body tissue to address sleep disordered breathing. At least some examples of such neurostimulation are later described and illustrated in association with FIGS. 3A-29. In some examples, therapy device 70 comprises an external therapy device, such as a device to provide airflow therapy (e.g. Continuous Positive Airway Pressure—CPAP) to address the sleep disordered breathing.

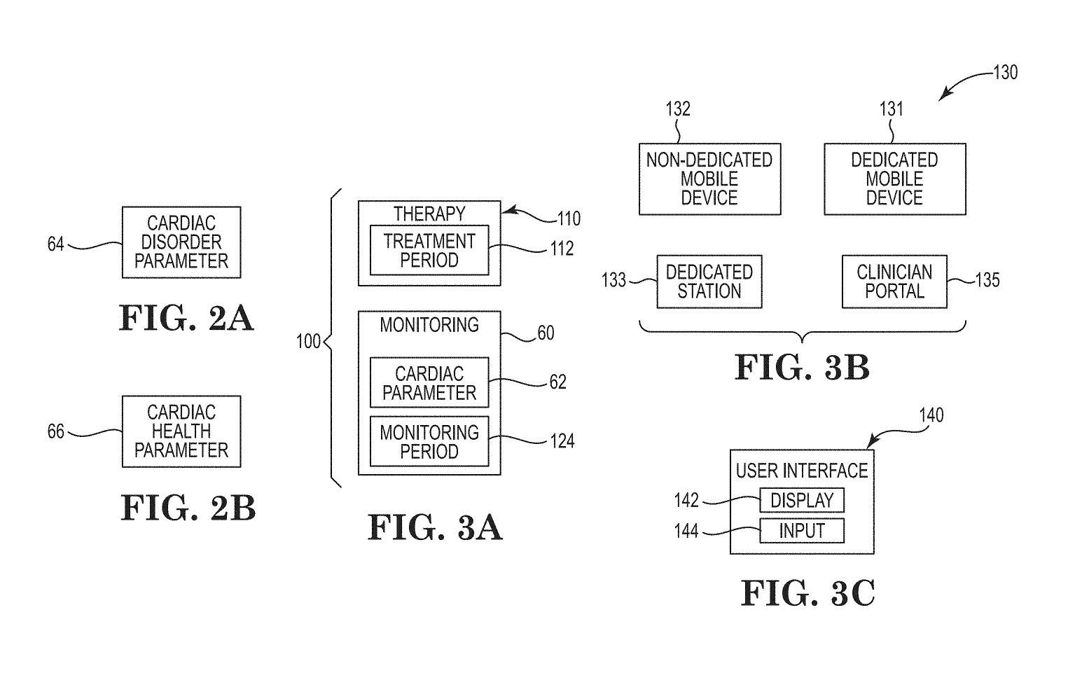

In some examples, monitoring resource 60 monitors a cardiac parameter 62 regarding the patient. In some examples, the cardiac parameter 62 is indicative of cardiac disorders as represented by cardiac disorder parameter 64 in FIG. 2A. In some examples, cardiac parameter 62 is indicative of cardiac health, as represented by cardiac health parameter 66 in FIG. 2B. In some examples, cardiac parameter 62 is indicative of both at least some aspects of cardiac disorders and at least some aspects of cardiac health.

In some examples, arrangement 50 enables treating the patient's sleep disordered breathing while also monitoring the patient for cardiac parameters. As more fully described later, such monitoring enables determining positive indications (e.g. enhanced cardiac health) and/or negative indications (e.g. evidence of cardiac disorders). In some examples, the indications regarding cardiac parameters may be short term, and in some examples, the indications regarding cardiac parameters may occur over the long term.

In some examples, monitoring resource 60 is implemented as monitoring resource 60 in association with a therapy manager 110 as shown in FIG. 3A, according to one example of the present disclosure.

In some examples, as represented by arrangement 100 in FIG. 3A, via therapy manager 110, treatment of sleep disordered breathing (via therapy device 70) may occur according to a treatment period 112 in FIG. 3A, while the cardiac parameter 62 may be monitored and/or evaluated via monitoring resource 60 according to a monitoring period 124 separate from, and independent of, the treatment period 112.

In general terms, the treatment period 112 refers to a time period during which treatment or therapy occurs. For instance, because sleep disordered breathing is generally associated with sleep periods of the patient, in some examples the treatment period 112 coincides with a daily sleep period of the patient. In some instances, the daily sleep period is identified via sensing technology which detects motion, activity, posture, position of the patient, as well as other indicia, such as heart rate, breathing patterns, etc. In some instances, the daily sleep period is selectably preset, such from 10 pm to 6 am or other suitable times.

However, in some examples, the treatment period 112 could be implemented intermittently, such as every other day or every third day, and the like. Moreover, in some examples, the treatment period 112 can be shorter or longer than the sleep period of the patient.

In some examples, commencement of a treatment period 112 does not necessarily mean that continuous stimulation is applied during the treatment period 112. Rather, various stimulation protocols can be implemented during a treatment period 112. In some implementations, the stimulation protocol includes stimulating pertinent body tissues (e.g. upper-airway-related body tissues) upon identification of a fiducial from a respiratory waveform and/or other information sensed at the patient, wherein the fiducial may be indicative of sleep disordered breathing.

In some instances, stimulation is generally synchronized with inspiration.

In some instances, whether or not stimulation is synchronized with inspiration, stimulation is triggered in association with at least one of a beginning of inspiration, an end of inspiration, a beginning of expiration, and/or an end of expiration.

In some instances, initiation, termination, and/or duration of stimulation are based on a sensed respiratory waveform but are not synchronized relative to each inspiratory phase.

In some examples, a stimulation protocol includes stimulating pertinent body tissues without sensing respiratory information and/or without being synchronized relative to inspiration.

In some of these examples, the monitoring period 124 may have a duration on the same order of magnitude as the treatment period 112. For instance, if the treatment period 112 occurs daily (or every other day, every third day, etc.), the monitoring period 124 may be daily or some number (e.g. 2, 3, 4, 5, 6, 7) of days.

However, in some examples, the monitoring period 124 may have a duration on a different order of magnitude than the treatment period 112. In some examples, the monitoring period 124 has a duration that is at least one order of magnitude greater than the duration of the treatment period 112. Accordingly, the monitoring period 124 may be ten days, two weeks, several weeks, a month, a quarter, one-half year, a year, etc.

In some examples, a duration of the monitoring period 124 is based on each particular diagnosable cardiac disorder. In particular, the duration of the monitoring period 124 is selected to correspond to a period of time by which one can observe indicia of the absence, presence, increase, or decrease of the particular cardiac disorder.

In some examples, a duration of the monitoring period 124 is based on each particular cardiac health parameter. In particular, the duration of the monitoring period 124 is selected to correspond to a period of time by which one can observe indicia of the absence, presence, increase, or decrease of the particular cardiac health.

In some examples, a monitoring resource 60 comprises part of or is incorporated within the therapy device 70. As such, some example monitors may sometimes be referred to as being “on board” the therapy device 70. In some examples, monitor is external to the therapy device 70 but is coupled to and/or in communication with therapy device 70. In some examples, monitoring resource 60 is dedicated to monitoring and/or evaluating the cardiac parameter 62. In some examples, monitoring and/or evaluating the cardiac parameter 62 are just some functions of multiple functions of monitoring resource 60. In some examples, monitoring resource 60 may support managing at least some general operations of therapy device 70.

In some examples, monitoring resource 60 cooperates with and/or forms part of a control portion, such as but not limited to, control portion 880 as later described in association with at least FIG. 23. In some examples, the monitoring resource 60 at least partially fulfills the role of engine 885 in FIG. 23. In some examples, monitoring resource 60 completely fulfills the role of engine 885 in control portion 880 (FIG. 23).

In some examples, therapy manager 110 in FIG. 3A cooperates with and/or forms part of a control portion, such as but not limited to, control portion 880 as later described in association with at least FIG. 23. In some examples, the therapy manager 110 (FIG. 3A) at least partially fulfills the role of engine 885 in FIG. 23. In some examples, therapy manager 110 completely fulfills the role of engine 885 in control portion 880 (FIG. 23).

In some examples, both monitoring resource 60 and therapy manager 110 work together in a complementary manner to at least partially fulfill the role of engine 885 of control portion 880 in FIG. 23.

With this general arrangement of system 50 in FIG. 1 in mind, it will be understood that at least some implementations associated with FIGS. 3B-23 provide more specific examples of various implementations and details regarding the operation and interaction of at least some aspects of monitoring resource 60 and/or therapy device 70.

FIG. 3B is block diagram schematically representing an array 130 of access tools 131-135, according to one example of the present disclosure. FIG. 3C is a block diagram schematically representing user interface 140, according to one example of the present disclosure. In some examples, at least some of the access tools 131-135 include user interface 140.

In some examples, user interface 140 comprises a user interface or other display that provides for the simultaneous display, activation, and/or operation of at least some of the various components, elements, engine, functions, parameters, features, and attributes of monitoring resource 60 and/or therapy manager 110 and/or control portion 880 (FIG. 23). In some examples, at least some portions or aspects of the user interface 140 are provided via a graphical user interface (GUI). In some examples, as shown in FIG. 3C, user interface 140 includes display 142 and input 144.

With further reference to FIG. 3B, in some examples the access tools comprise a mobile device 131 dedicated to facilitating operation of and/or monitoring operation of at least some aspects of therapy device 70 (FIG. 1). In some instances, at least some components of monitoring resource 60 and/or therapy manager 110 reside in the dedicated mobile device 131. In some instances, dedicated mobile device 131 may be embodied as or referred to as a patient remote, patient programmer, or patient controller.

In some examples, the access tools in FIG. 3B comprise a mobile device 132 not dedicated to, but capable of, facilitating operation of and/or monitoring operation of at least some aspects of monitoring resource 60 and/or therapy device 70 (FIG. 1). In some instances, at least some components of monitoring resource 60 and/or therapy manager 110 may be stored in the non-dedicated mobile device 132. In some instances, non-dedicated mobile device 132 may be embodied as or referred to as a smart phone, tablet, phablet, notebook computer, watch phone, etc. In some examples, at least some components of monitoring resource 60 and/or therapy manager 110 may be arranged as a function, widget, and/or application (i.e. mobile app), etc. on the non-dedicated mobile device 132. In some instances, the non-dedicated mobile device 132 may be used for functions (e.g. phone, computing, web browsing, texting, etc.) separate from, and independent of, operation of monitoring resource 60 and/or therapy manager 110.

In some examples, any one of the mobile devices 131, 132 and dedicated station 133 may include at least one of the sensors as later described in association with FIGS. 11-12 and/or may receive at least some of information 300 (FIG. 9).

For instance, in some examples, many commercially available non-dedicated mobile devices 132 include photographic and/or video recording capabilities which can be used to take still and/or moving images of a patient before, during, and after sleep. In some examples, this imaging functionality is embodied in image sensor 419 in FIG. 12. Similarly, in some examples, the commercially available, non-dedicated mobile devices 132 may include audio recording equipment, which can record snoring or other breathing sounds, patient activity, and sounds in the patient's sleep environment. In some examples, this audio functionality is embodied in acoustic sensor 418 in FIG. 12.

In some examples, dedicated station 133 comprises any device or instrument locatable within a patient's sleep environment, and which is dedicated to facilitating operation of and/or monitoring operation of at least some aspects of therapy device 70 (FIG. 1). In some instances, at least some components of monitoring resource 60 and/or therapy manager 110 reside in the dedicated station 133. In some instances, dedicated station 133 may be embodied as or referred to as a patient remote, patient programmer, or patient controller. In some examples, dedicated station 133 comprises at least some of substantially the same features and attributes as mobile devices 131, 132.

In some examples, clinician portal 135 facilitates operation of and/or monitoring operation of at least some aspects of therapy device 70 (FIG. 1) by a clinician. In some instances, at least some components of monitoring resource 60, and/or therapy manager 110 are accessible via clinician portal 133. In some instances, clinician portal 133 may be embodied as or referred to as a clinician remote, clinician programmer, or clinician controller.

In some examples, regardless of the form of the access tool, at least some of the features and functions of monitoring resource 60, and/or therapy manager 110 are accessible via a web-centric model.

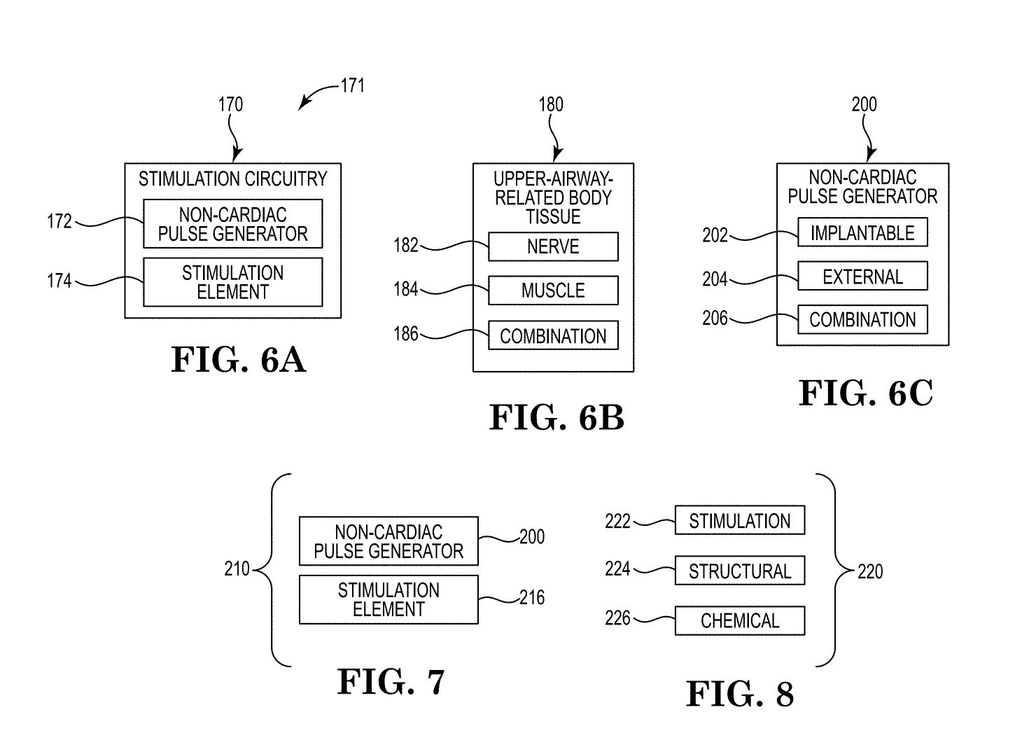

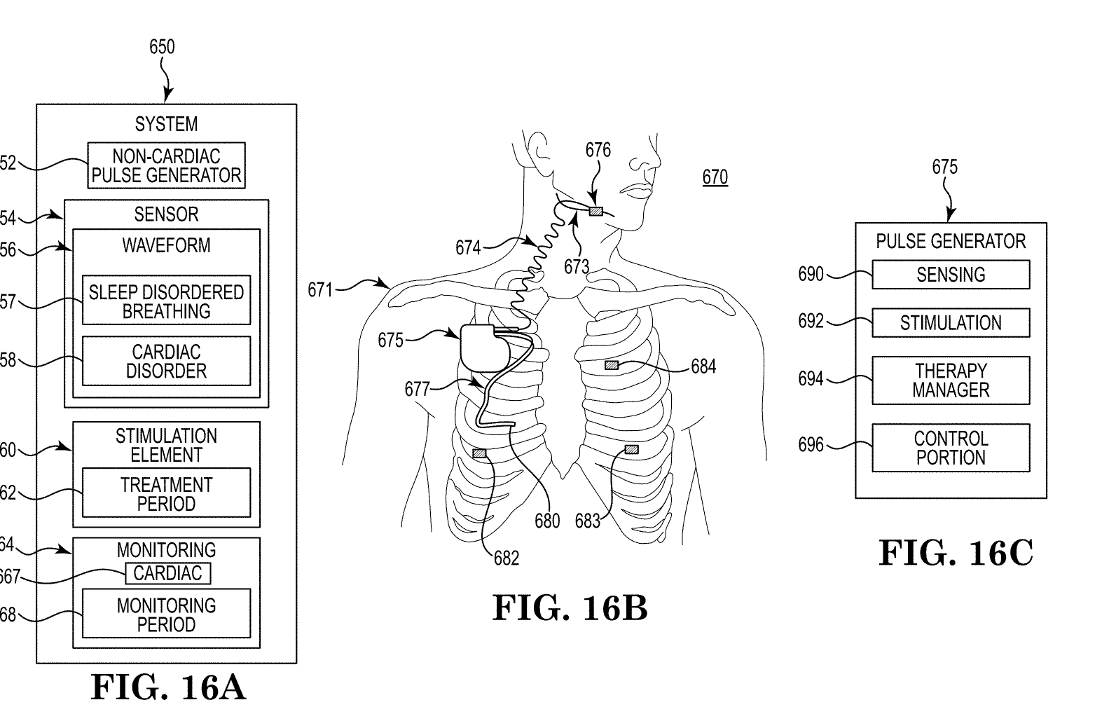

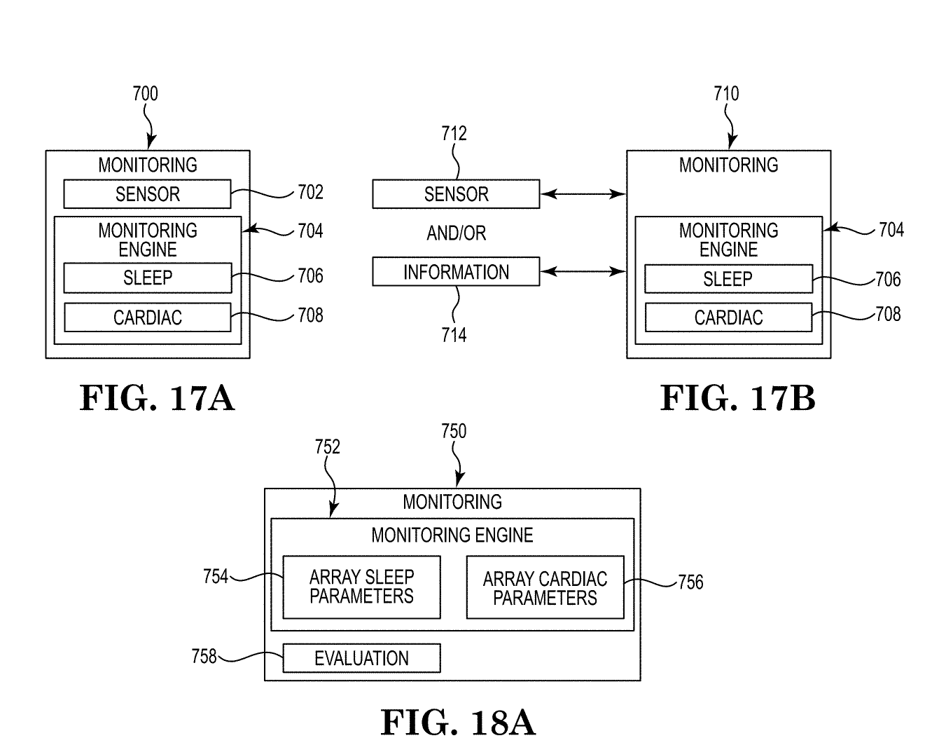

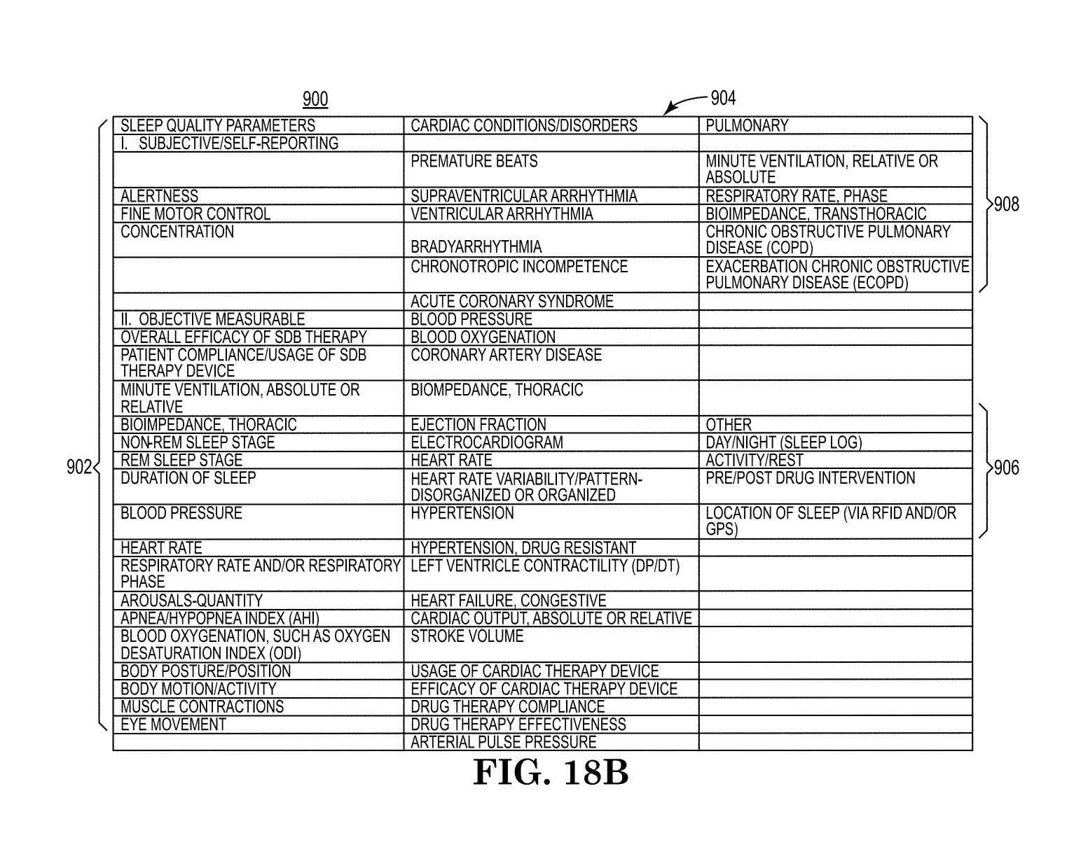

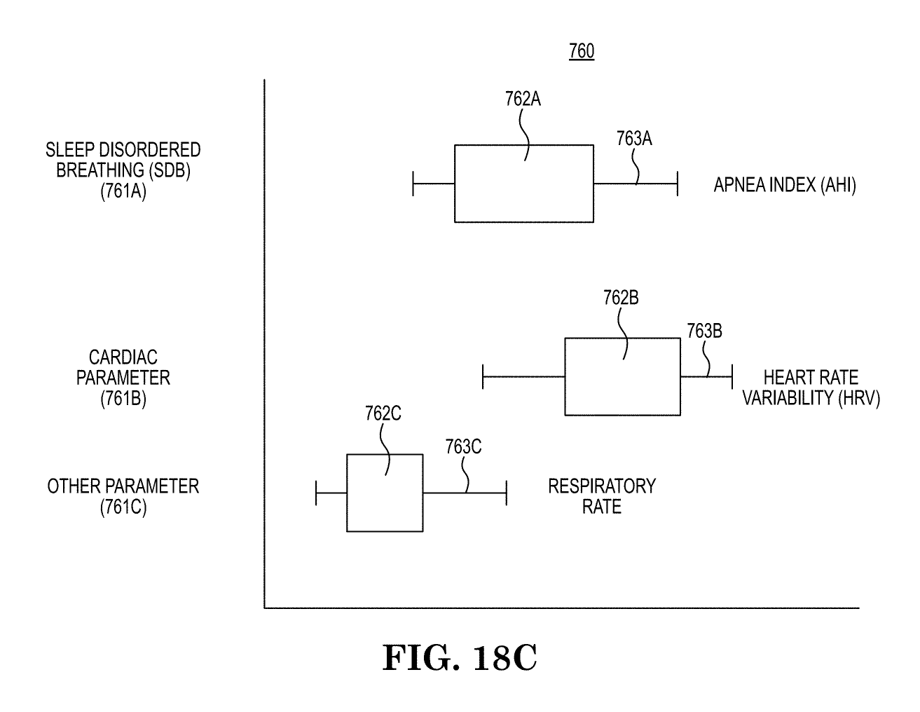

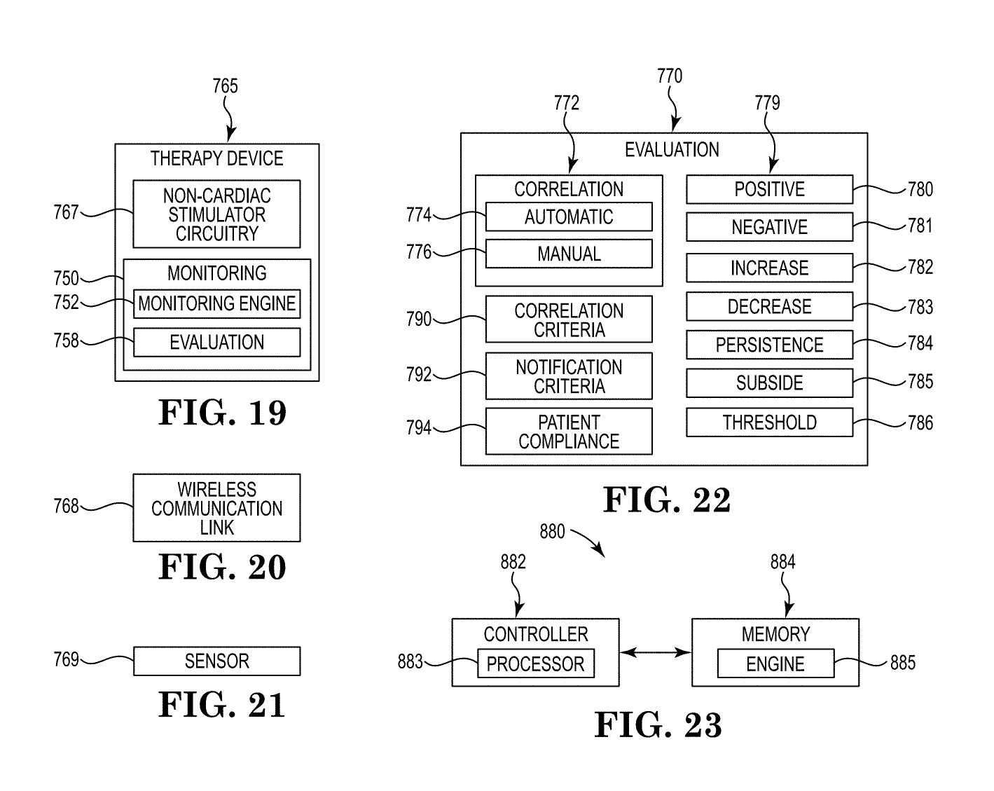

In some examples, at least one of the access tools 131-135 for facilitating operation of monitoring resource 60 and/or therapy manager 110 are cooperable with the therapy devices/systems (e.g. 170 in FIG. 6A; 340 in FIG. 10A; 350 in FIG. 10B; 650 in FIG. 16A; 670 in FIGS. 16B-16C; 765 in FIG. 19). In some examples, access tools 131-135 employed in association with monitoring resource 60 and/or therapy manager 110 may include or be cooperable with determination engine (e.g. 570 in FIG. 15C; 704 in FIG. 17A; and 752 in FIG. 18A), cooperable with monitoring resources (e.g. 700 in FIG. 17A; 710 in FIG. 17B; 750 in FIG. 18A) and/or cooperable with evaluation engine (770 in FIG. 22), as described in the examples throughout the present disclosure.

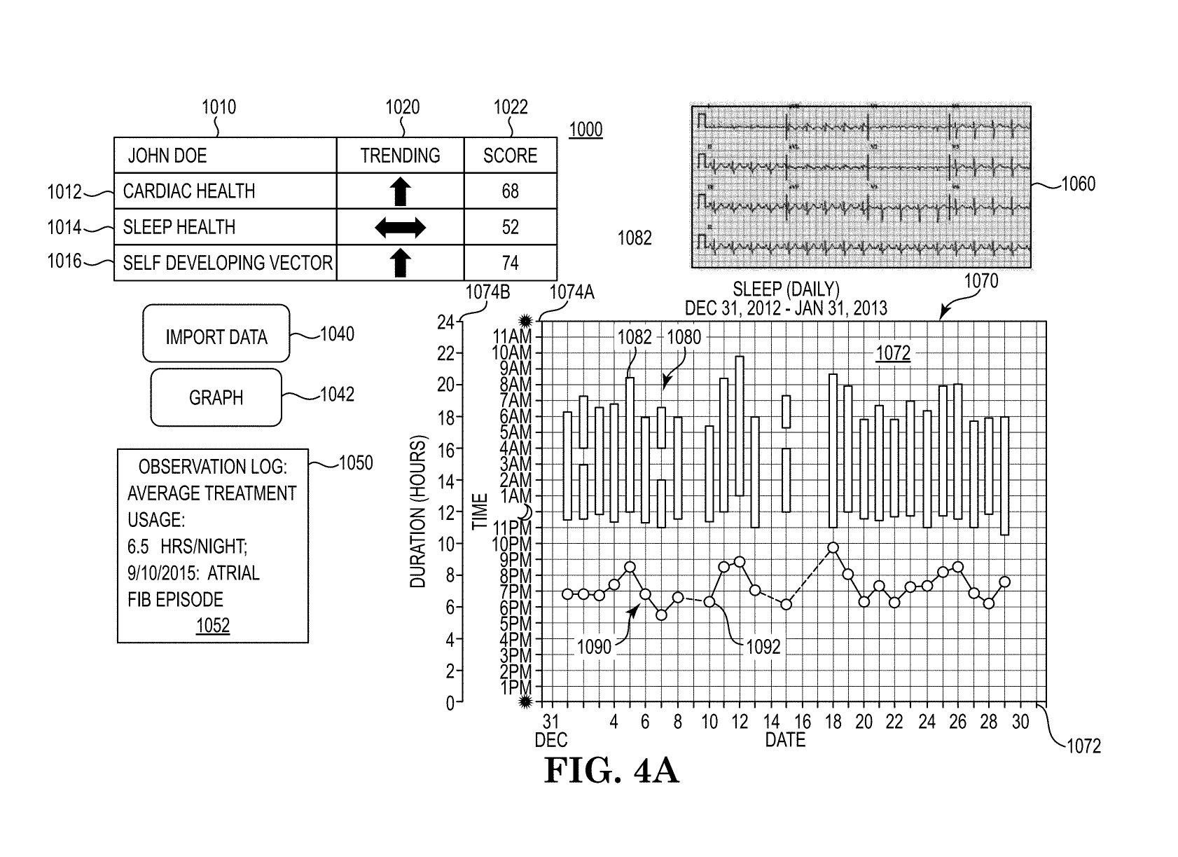

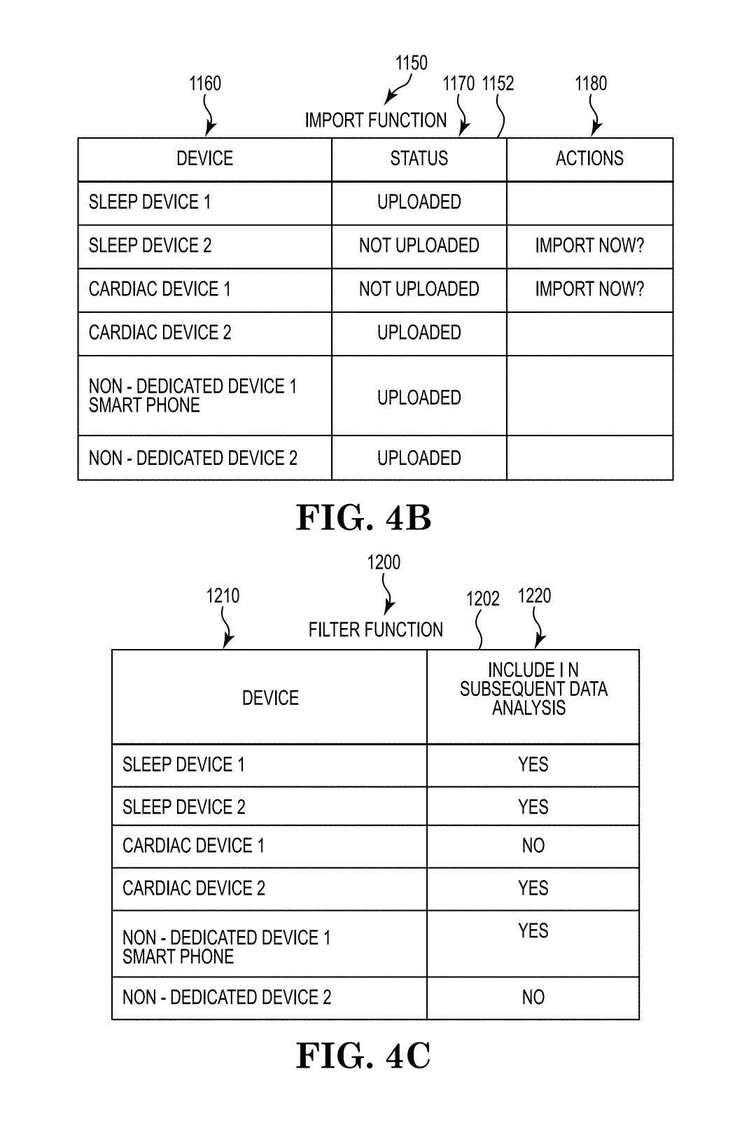

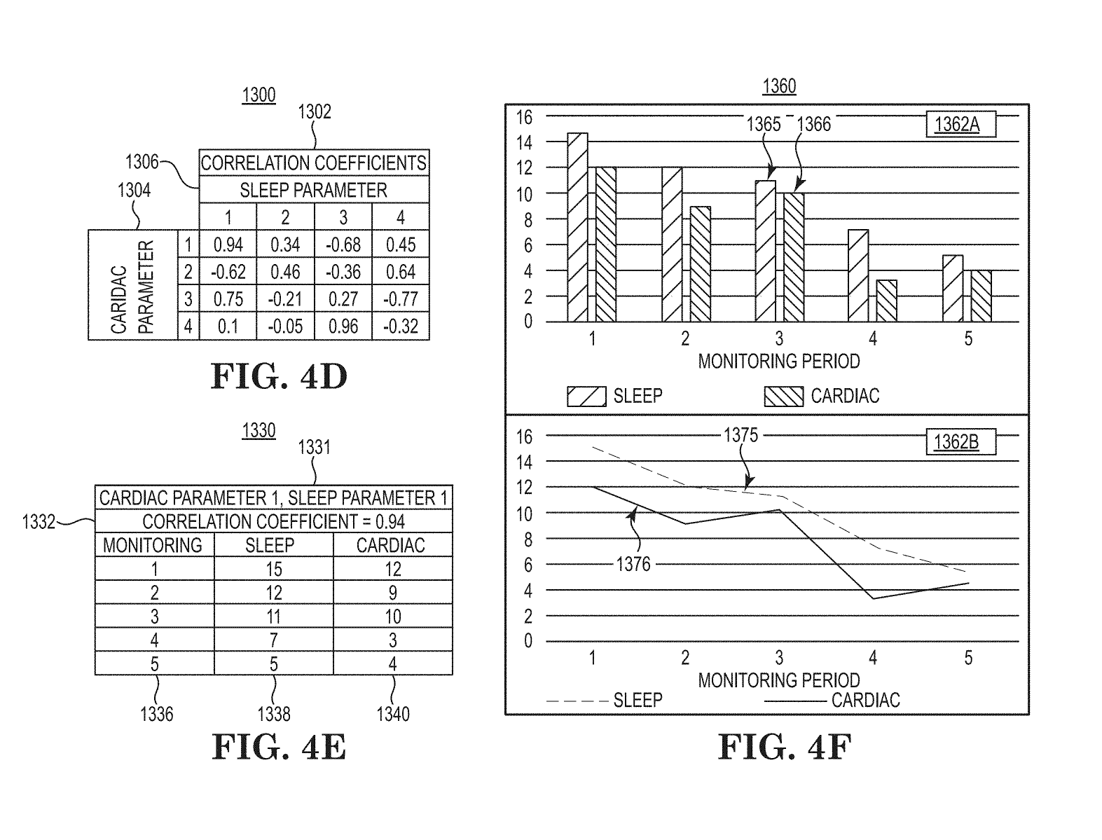

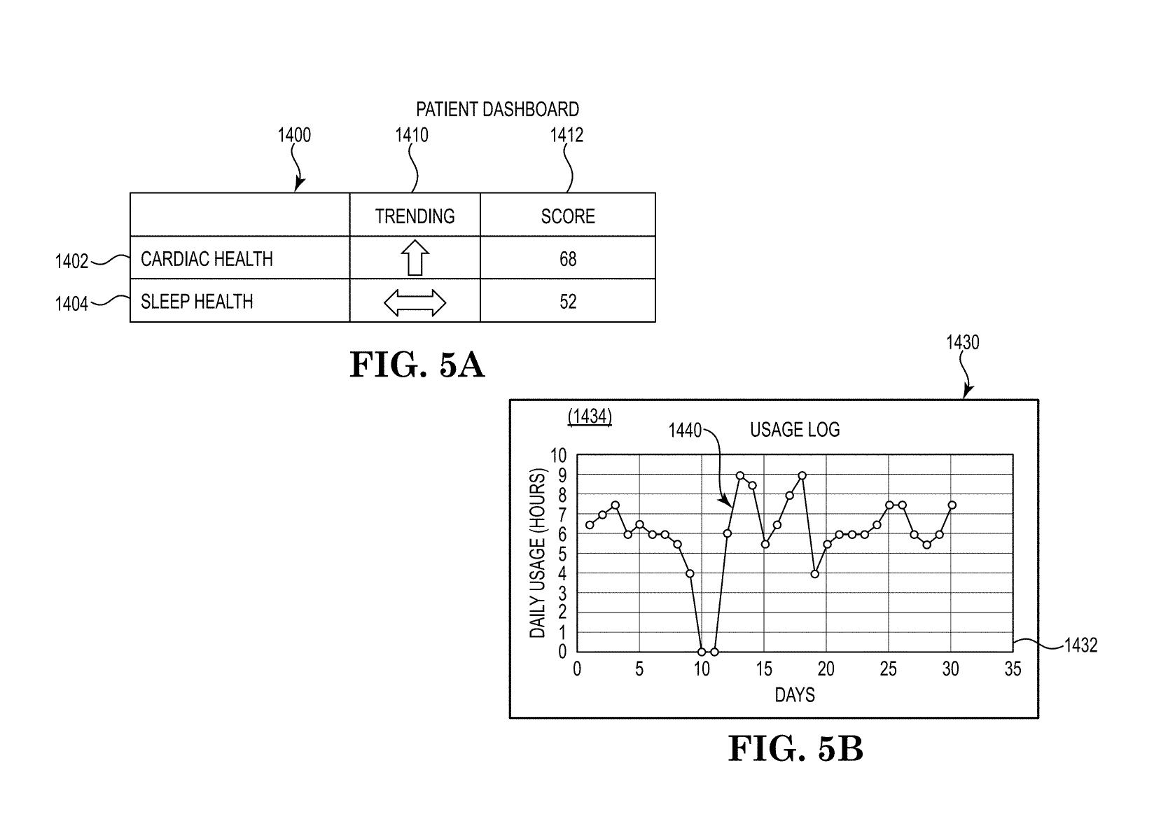

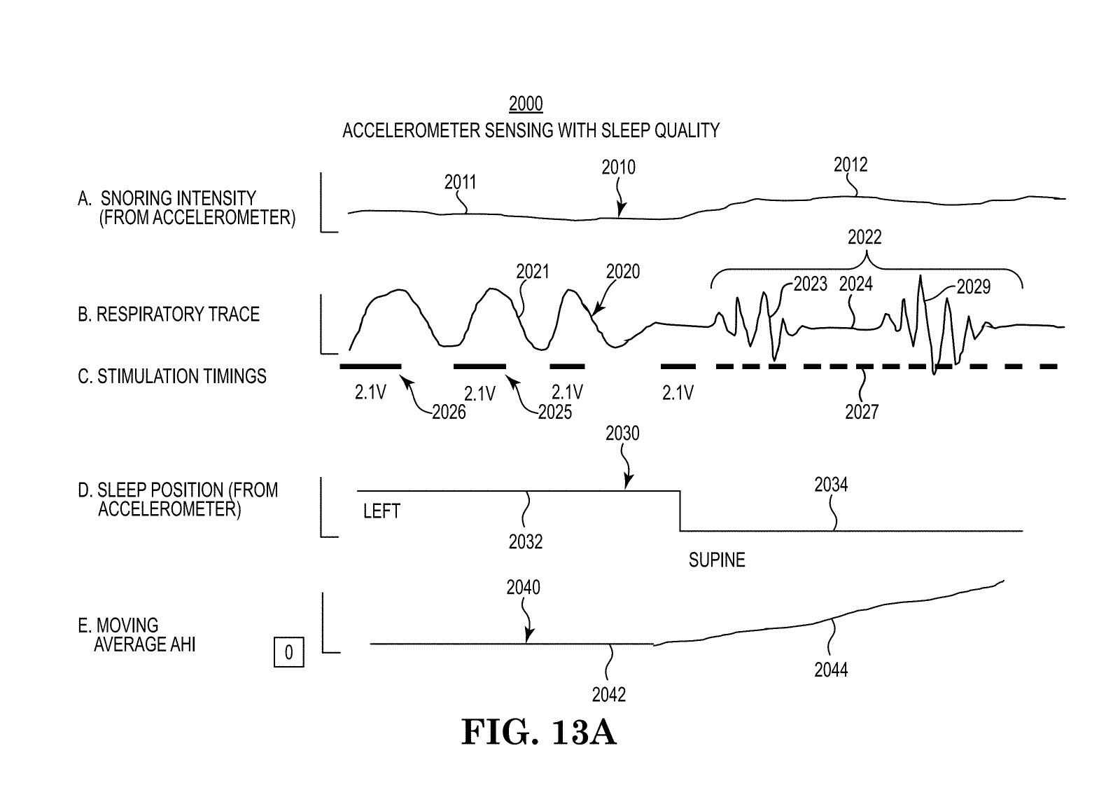

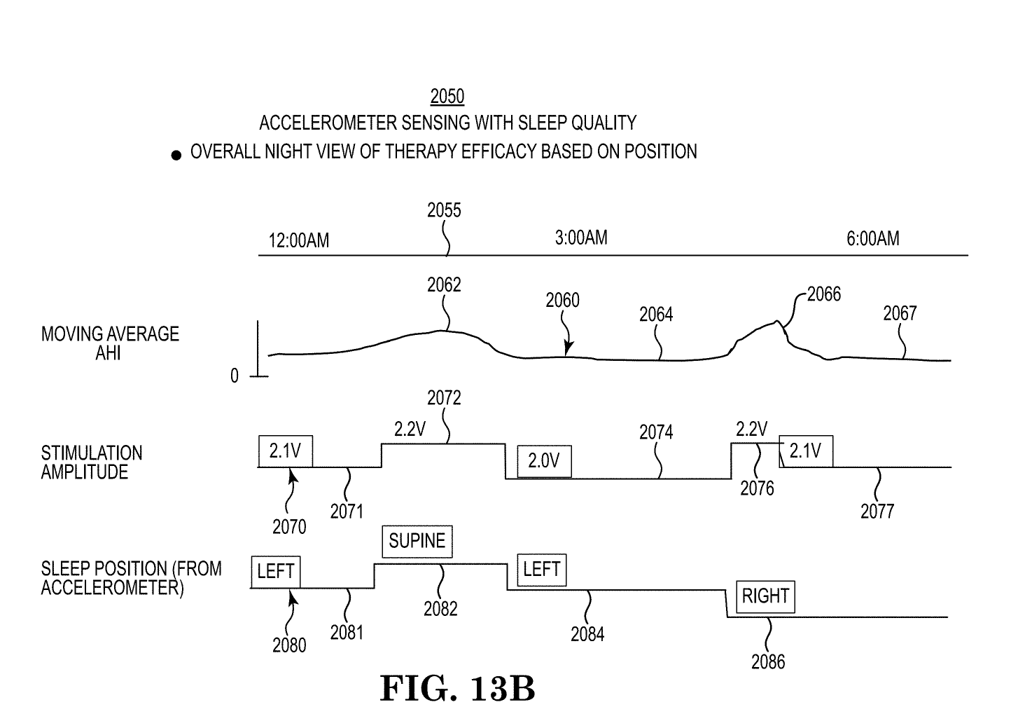

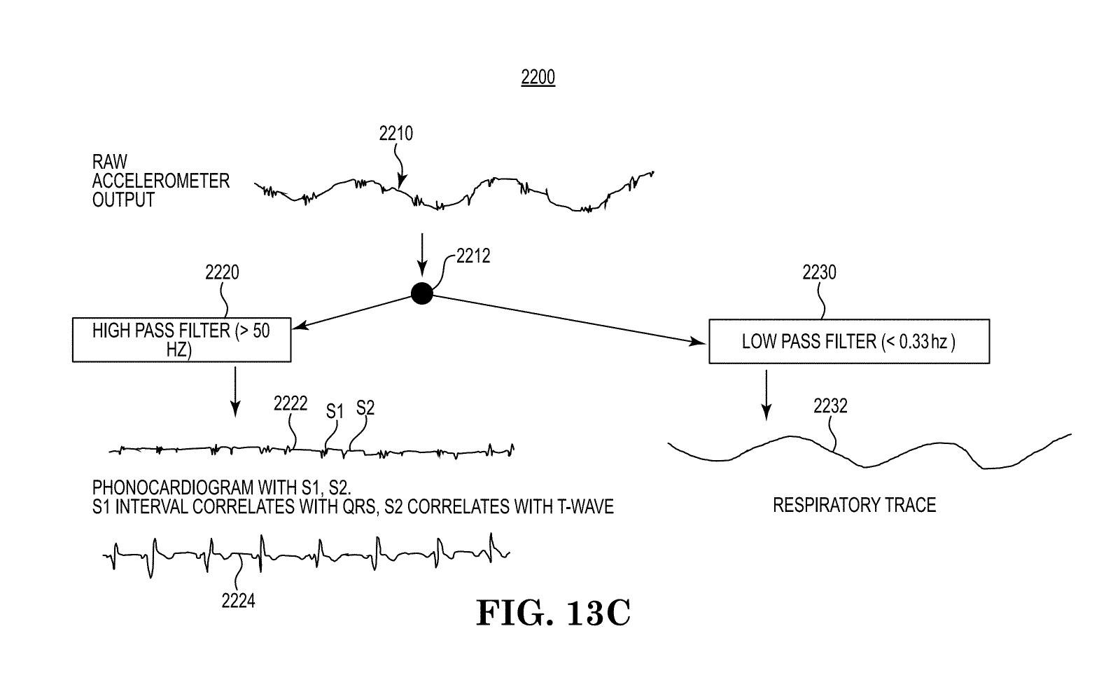

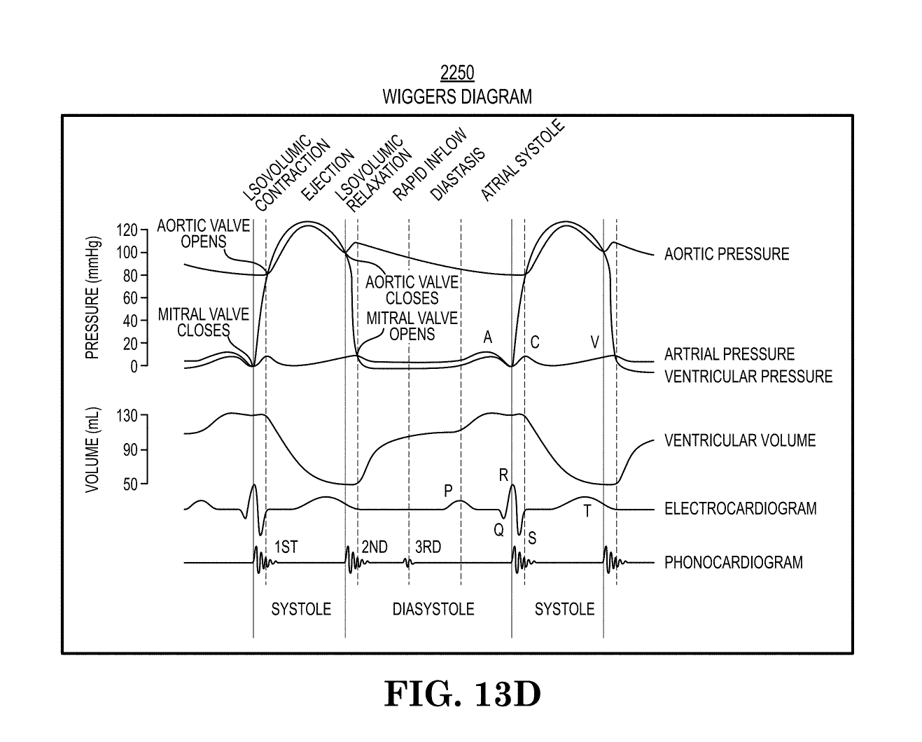

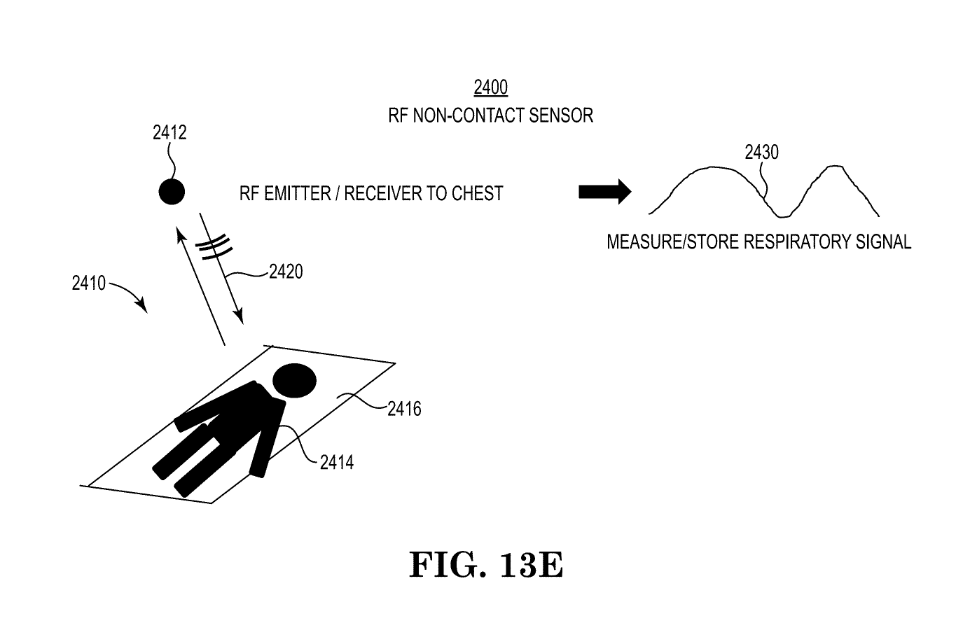

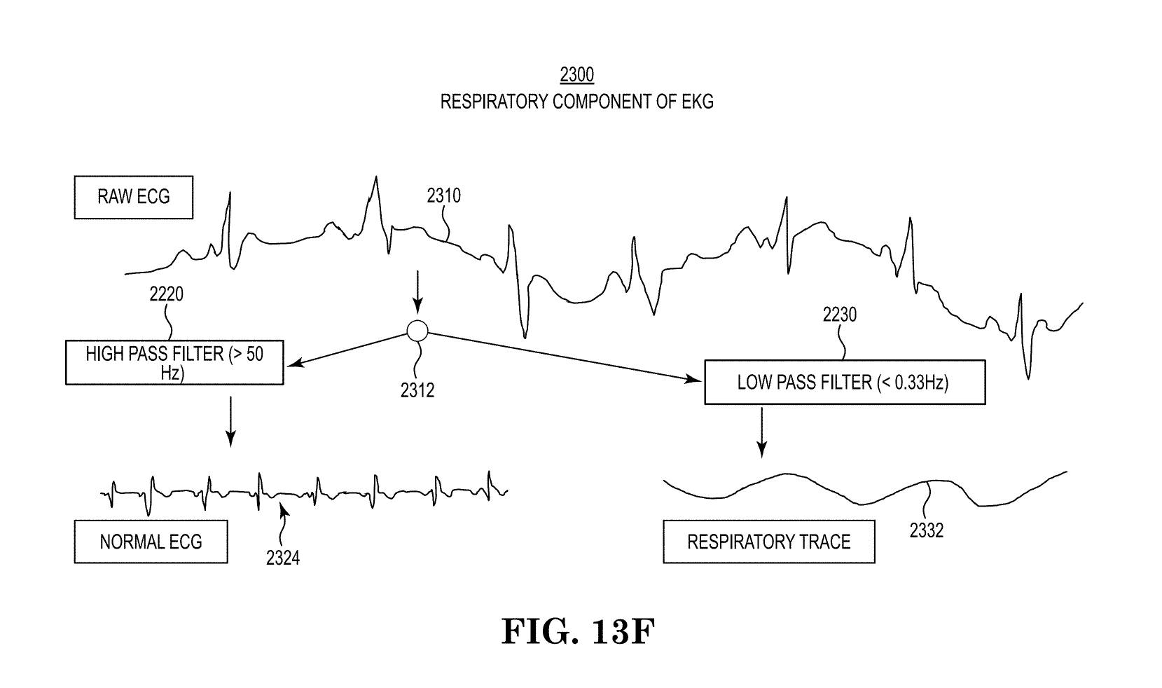

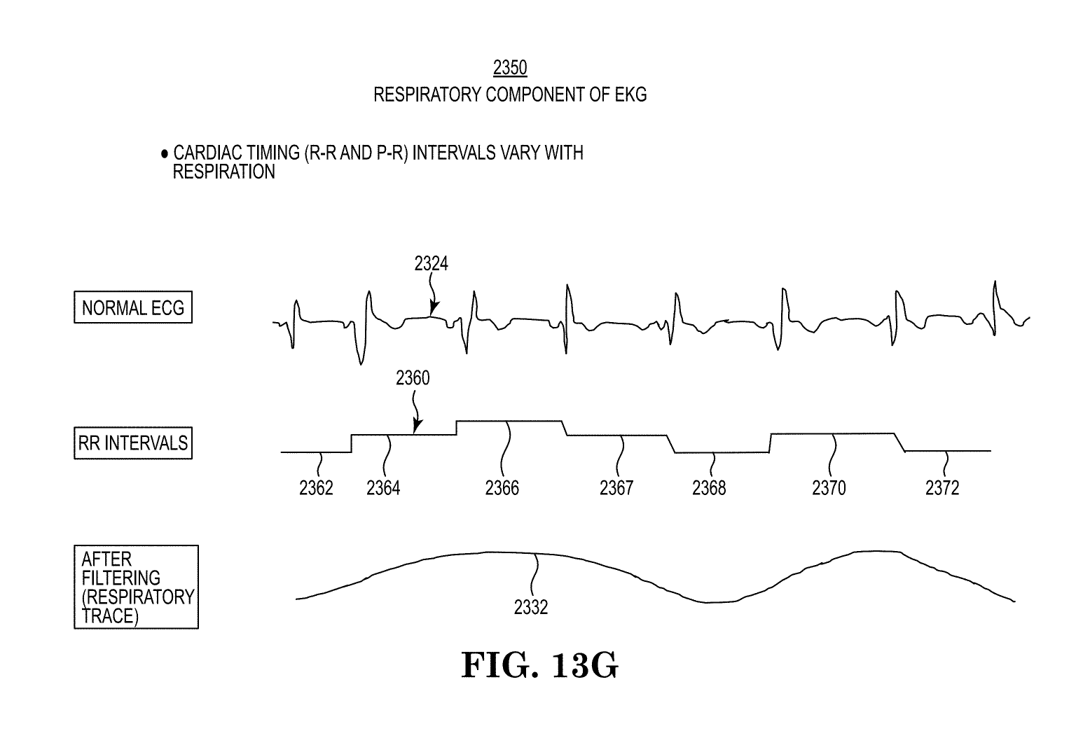

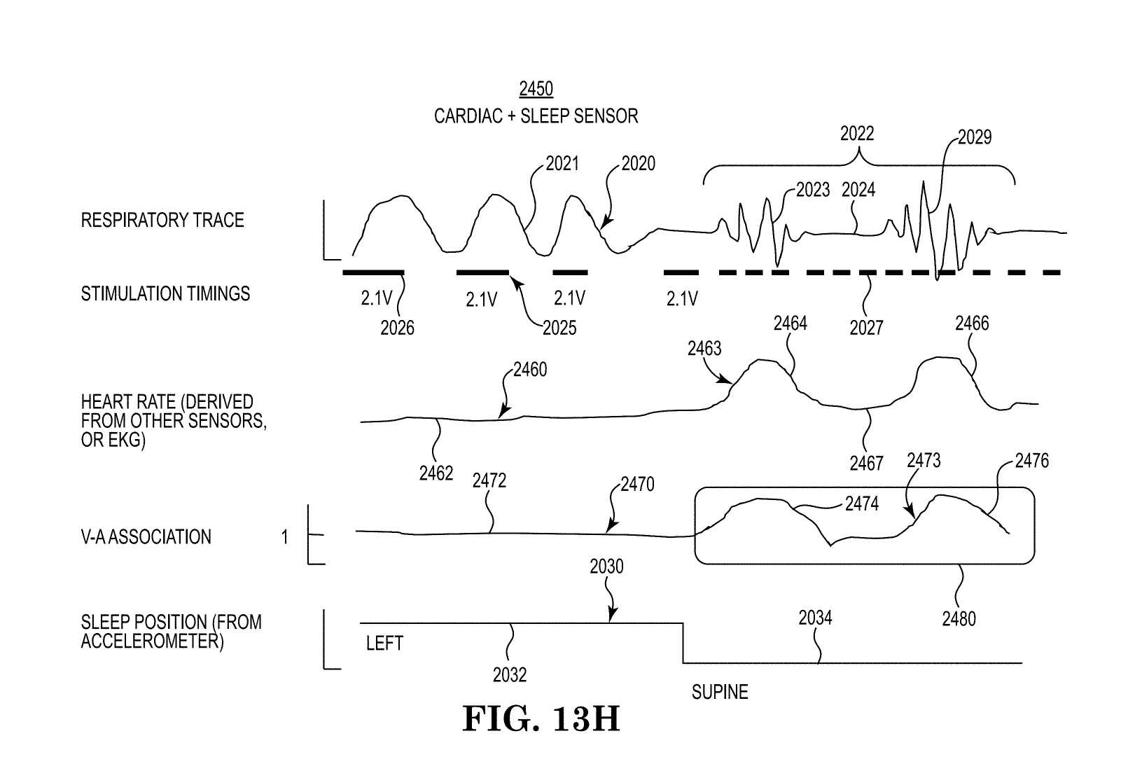

FIGS. 4A-5B include diagrams schematically representing at least some instances of a user interface associated with cardiac-related monitoring according to at least some examples of the present disclosure. It will be understood that in some examples, user interface portions represented in one Figure may be combined in a complementary manner with user interface portions in another Figure or user interface portions among several Figures. Likewise, in some examples, some user interface portions represented in FIGS. 4A-5B may be combined in a complementary manner with some user interface portions throughout various examples in at least FIGS. 13A-13I.

FIG. 4A is a diagram schematically representing one instance of a clinician user interface 1000, according to one example of the present disclosure. In some examples, a clinician user interface 1000 may include all of the components shown in FIG. 4A, while in some examples, a clinician user interface 1000 includes just some of the components shown in FIG. 4A.

In some examples, clinician user interface 1000 comprises at least some of substantially the same features as user interface 140 in FIG. 3C. In some examples, clinician user interface 140 may be accessed via at least one of the access tools 131-135 in FIG. 3B.

In some examples, clinician user interface 1000 displays information about a particular patient, and as such includes a patient table 1010 reporting several parameters 1012-1016 regarding a physiologic state of a patient. In some examples, parameters 1012-1016 include a cardiac parameter 1012, a sleep parameter 1014, and/or a self-developing vector 1016. It will be understood that greater or fewer than three parameters 1012-1016 can be monitored and displayed in table 1010.

Table 1010 further includes a trending column 1020, which indicates whether a particular parameter 1012-1016 is trending upward, downward, or is steady as represented by corresponding directional arrows. A score column 1022 indicates a score according to an alphanumeric scoring scale, which in some instances, indicates a relative value for a particular parameter 1012-1016. In some instances, an absolute value may be displayed.

As further shown in FIG. 4A, in some examples clinician user interface 1000 includes an import data function 1040 and/or a graph function 1042. In general terms, import data function 1040 initiates and/or controls importing of data from patient device(s) and/or other patient-related information databases. One example implementation of the import data function 1040 is later described and illustrated in association with at least FIG. 4B.

In some examples, clinician user interface 1000 includes an observation log element 1050 to display therapy-related information 1052. In some examples, the particular types of information displayed are selectable by the clinician and in some examples, the particular types of information are fixed by the device manufacturer.

As shown in FIG. 4A, in some examples, information 1052 includes average treatment usage, such as the number of hours per night that therapy was applied. In some examples, information 1052 includes episodal information, which may include episodes regarding obstructive sleep apnea, cardiac disorder episodes, and/or pulmonary episodes, etc. In the particular example shown in FIG. 4A, information 1052 includes a cardiac episode, such as an instance of atrial fibrillation along with the date of occurrence.

In some examples, the information 1052 may be hourly, daily, weekly, monthly, etc.

As further shown in FIG. 4A, in some examples clinician user interface 1000 includes a graph 1060 displaying physiologic information, such as but not limited to a cardiac waveform. In some examples, the cardiac waveform may reveal an episode indicative of a cardiac disorder. For instance, the cardiac waveform in graph may reveal a potential episode of atrial fibrillation (AF), and which may be noted in LOG 1050.

As further shown in FIG. 4A, in some examples a clinician user interface 1000 includes a graph 1070 displaying sleep information 1072. In some examples, such sleep information 1072 is plotted according to an x-axis 1072 representing a date and a first y-axis 1074A representing a time of day, and a second y-axis 1074B representing a duration (e.g. in hours).

In some examples, sleep information 1072 plotted on graph 1070 includes a series 1080 of daily sleep periods 1082, illustrating whether sleep is generally continuous or broken and the start time (e.g. about 11 pm) and end time (e.g. about 7 am) of the daily sleep period 1082 for a particular date. In some examples, sleep information 1072 plotted on graph 1070 includes a series 1090 of durations 1092 (e.g. 7.5 hours) of the daily sleep periods.

FIG. 4B is a table schematically representing aspects of an import function 1150 associated with a clinician user interface, according to one example of the present disclosure. In some examples, import function 1150 comprises at least some of substantially the same features and attributes as import data function 1040 in FIG. 4A. In some examples, import data function 1150 monitors and controls the importing of data from patient devices into clinician user interface 1000 and/or other clinician management tools. As further shown in FIG. 4B, import function 1150 can utilize a table 1152, which includes a device column 1160, a status column 1170, and an action column 1180. The device column 1160 lists which devices, by type and/or patient identity, for which therapy is to be monitored and/or evaluated via clinician user interface 1000. The status column 1170 lists an upload data status regarding each listed device, while the action column 1180 lists potential actions that can be taken regarding a particular listed device. It will be understood that import function 1150 includes the ability to add or remove devices from table 1152.

FIG. 4C is a table 1202 schematically representing aspects of a filter function 1200 associated with a clinician user interface, according to one example of the present disclosure. In some examples, filter function 1200 works in association with import function 1150 to facilitate a clinician decision whether data from a particular device (and therefore a particular patient) listed in column 1210 is to be included in subsequent data analysis, as indicated via column 1220.

In some examples, table 1152 or table 1202 comprise a displayable user interface or may form part of a clinician user interface 1000, such as upon engagement of the import data function 1040. In some examples, tables 1152, 1202 comprise at least some of substantially the same features and attributes as user interface 140 in FIG. 3C.

FIGS. 4D-4F provide further tables and graphs, which may form part of a clinician user interface, such as but not limited to clinician user interface 1000, and may comprise at least some of substantially the same features and attributes as user interface 140 in FIG. 3C.

FIG. 4D is a table 1300 schematically representing a correlation coefficient array 1302 for a plurality of sleep parameters 1306 and a plurality of cardiac parameters 1304, according to one example of the present disclosure. As shown in FIG. 4D, various sleep parameters (e.g. 1, 2, 3, 4) and various cardiac parameters (e.g. 1, 2, 3, 4) are mapped relative to each other and from which a correlation coefficient (e.g. 0.94) can be determined for each pairing of sleep parameters (e.g. 1) and cardiac parameters (e.g. 1). In some examples, the sleep parameters relate to aspects of sleep quality and one of the parameters also may represent an overall sleep quality parameter, which is a combination of various sleep parameters. In some examples, the cardiac parameters relate to aspects of cardiac disorder (or cardiac health) and one of the parameters in table 1330 also may represent an overall cardiac disorder parameter (alternatively, a cardiac health parameter), which is a combination of various cardiac parameters. The correlation coefficients facilitate identification of a relationship between various sleep parameters and cardiac parameters, such that a clinician may identify and monitor cardiac disorders (alternatively, cardiac health) in association with sleep parameters.

In some examples, the table 1300 in FIG. 4D provides one example implementation of the array 754 of sleep quality parameters and the array 756 of cardiac disorder parameters determined via determination engine 752, as later described in association with at least FIG. 18A.

FIG. 4E is a table 1330 schematically representing a correlation coefficient table for one sleep parameter and one cardiac parameter, according to one example of the present disclosure. Scores for a sleep parameter 1338 and scores for a cardiac parameter 1340 are determined through different monitoring periods 1336. In some examples, the overall correlation coefficient between sleep parameter 1 and cardiac parameter 1 is 0.94. Each monitoring period (e.g. 1, 2, 3, 4, 5, etc.) may be hourly, daily, weekly, monthly, quarterly, yearly, etc. In some examples, some monitoring periods may have a duration different than other monitoring periods within a group of monitoring periods.

FIG. 4F is a diagram 1360 including a pair of graphs 1362A, 1362B schematically representing the information in the table of FIG. 4E, with graph 1362A including an array 1365 of the sleep parameter scores (1338 in FIG. 4E) and an array 1366 of the cardiac parameter scores (1340 in FIG. 4E). Graph 1362B maps the same parameters according to a sleep parameter trend 1375 and a cardiac parameter trend 1376. In one aspect, the generally matching downward trajectory of both parameters may be interpreted, in some examples, that as the sleep parameter declines so does the cardiac parameter. Depending on whether the sleep parameter is determined to be a positive trait or a negative trait and depending on whether the cardiac parameter is determined to be a positive trait or a negative trait, the matching trendlines may indicate different types of association (e.g. positive, negative) between the particular sleep parameter and the particular cardiac parameter.

In some examples, at least one of the different types of sleep parameters (e.g. quality or disorder) may correspond to obstructive-sleep-apnea (OSA)-related parameters. In some examples, the OSA-related parameters may comprise a number of obstructive sleep apnea events or a severity of obstructive sleep apnea behavior.

FIGS. 5A-5B provide patient-oriented tables and graphs, which may form part of a patient user interface, which may comprise at least some of substantially the same features and attributes as user interface 140 in FIG. 3C. In some examples, the aspects of a patient user interface represented by FIGS. 5A-5B are accessible via one of the access tools 131-135 represented in FIG. 3B.

FIG. 5A is a table 1400 schematically representing information regarding a cardiac parameter and a sleep parameter in one instance of a patient user interface, according to one example of the present disclosure. In some examples, table 1400 comprises at least some of substantially the same features and attributes as table 1010 (FIG. 4A) except in a simplified form that omits self-developing vector 1016. However, table 1400 does include a trend column 1410 and a score column 1412 by which a cardiac parameter 1402 (e.g. health or disorder) and a sleep parameter 1404 (e.g. quality or disorder) may be monitored.

FIG. 5B is a graph 1430 schematically representing information regarding a sleep parameter in one instance of a patient user interface, according to one example of the present disclosure. While graph 1430 can take many forms and can represent many different kinds of patient information, in some examples graph 1430 maps instances of sleep duration (e.g. y-axis 1434) relative to days (x-axis 1432), thereby providing a map of trends. With or without other sleep parameters, sleep duration may provide an indication of sleep quality.

FIG. 6A is a block diagram schematically representing a therapy device 171, according to one example of the present disclosure. In some examples, therapy device 171 comprises at least some of substantially the same features and attributes as therapy device 70 (FIG. 1), and in some examples, therapy device 171 may act as therapy device 70 in FIG. 1.

As shown in FIG. 6A, therapy device 171 comprises stimulation circuitry 170 which includes a non-cardiac pulse generator 172 and a stimulation element 174. In some examples, the non-cardiac pulse generator 172 and stimulation element 174 are formed as a single unit, which can be multiple, separate components joined together or which may be a monolithic formation including both the non-cardiac pulse generator 172 and the stimulation element 174.

The therapy device 171 enables electrically stimulating upper-airway-related body tissue 180, such as schematically represented in FIG. 6B. In general terms, upper-airway-related body tissue comprises any body tissue which can affect the function and/or operation of the upper airway, and which can be stimulated in some form to efficaciously address sleep disordered breathing, such as via restoring upper airway patency or via other physiologic mechanisms. In some examples, the body tissue includes nerve(s) 182, muscle 184, or a combination 186 of nerve and muscle. In some examples, the particular nerve(s) 182 and/or muscle(s) 184, upon stimulation, restore patency of the upper airway and thereby alleviate obstructive sleep apnea.

FIG. 6C is a block diagram schematically representing a non-cardiac pulse generator 200, according to one example of the present disclosure. In some examples, non-cardiac pulse generator 200 may act as a non-cardiac pulse generator (e.g. 172 in FIG. 6A; 200 in FIG. 7; 652 in FIG. 16A), and/or may be incorporated into therapy device (e.g. 70 in FIG. 1; 340 in FIG. 10A; 350 in FIG. 10B; 765 in FIG. 19).

In some examples, the non-cardiac pulse generator 200 includes entirely implantable components 202. In some examples, the non-cardiac pulse generator 200 includes some implantable components 202 and some external components 204 to form a combination 206. In some examples, the non-cardiac pulse generator 200 is wholly external to the patient's body.

In general terms, the non-cardiac pulse generator 200 can generate electrical signals deliverable through a stimulation element (e.g. 174 in FIG. 6A; 216 in FIG. 7) suitable for exciting body tissue 180 to restore airway patency. In some examples, the signals are adapted to directly stimulate upper-airway-related muscles 184 and/or to stimulate nerves 182 innervating such muscles 184. In some examples, such as the case of obstructive sleep apnea, the nerves 182 may include (but are not limited to) the nerve 182 and the muscles 184 related to causing movement of the tongue and related musculature to restore airway patency. In some examples, the nerves 182 may include (but are not limited to) the hypoglossal nerve and the muscles 184 may include (but are not limited to) the genioglossus muscle.

In some examples, the non-cardiac pulse generator 200 forms part of the INSPIRE® Upper Airway Stimulation system, available from Inspire Medical, Inc. of Maple Grove, Minn. In some examples, the pulse generator 200 comprises a pulse generator available from other vendors.

Further examples of the non-cardiac pulse generator 200 are later described in association with at least FIGS. 7 and 16A.

FIG. 7 is a block diagram schematically representing components of a therapy device 210, according to one example of the present disclosure. As shown in FIG. 7, therapy device 210 includes a non-cardiac pulse generator 200 and stimulation element 216. In some examples, pulse generator 200 includes at least some of substantially the same features and attributes as pulse generator 200, as previously described in association with at least FIG. 6C. In some examples, stimulation element 216 comprises at least some of substantially the same features and attributes as stimulation element 174, as previously described in association with at least FIG. 6A.

In general terms, therapy device 210 enables stimulation of upper-airway-related body tissue 180 (FIG. 6B). In some examples, the pulse generator 200 and stimulation element 216 are not necessarily physically co-located. However, in some examples pulse generator 200 and stimulation element 215 may be co-located in close physical proximity to each other. For instance, in some examples, both the pulse generator 200 and stimulation element may be located in proximity to a target stimulation site. However, in some examples, the stimulation element 216 is located at or near a target stimulation site while the pulse generator 200 is located remotely from the target stimulation site. In some examples, pulse generator 200 and/or stimulation element 216 include wireless communication elements to enable wireless communication therebetween.

In some examples, the pulse generator 200 is implanted within a pectoral region and the stimulation element 216 comprises a cuff electrode coupled relative to a nerve, such as the hypoglossal nerve. Further details regarding such examples are provided later in association with at least FIGS. 16A-16B.

In one aspect, pulse generator 200 is at least electrically coupled relative to stimulation element 216 and is also physically coupled relative to stimulation element 216, such as via a lead extending between the pulse generator 200 and the stimulation element 200. However, in some examples, pulse generator 200 is physically coupled relative to stimulation element 216 via structures other than an electrical lead.

FIG. 8 is a block diagram 220 schematically representing various modalities of restoring airway patency, according to one example of the present disclosure. As shown in FIG. 8, such modalities include stimulation 222, structural 224, and chemical 226. The stimulation modality 222 is described throughout the present disclosure in association with at least FIGS. 6A, 6C, 7, 10A-10B, 16A-16B, and 19. Structural modality 224 comprises installing a structural component within the upper airway or nearby bodily structures to at least partially modify or influence the patency of the upper airway. In some examples, the various modalities 222, 224, and 226 may be implemented in different combinations, such as but not limited to, employing both a stimulation modality 222 and a structural modality 224.

FIG. 9 is a block diagram schematically representing patient information 300, according to one example of the present disclosure. In some examples, patient information 300 includes respiratory information 302, cardiac information 304, sleep quality information 306, sleep disordered breathing (SDB) information 308, and/or other information 310. In some examples, various combinations of information 302, 304, 306, 308, and 310 may be used, as represented via combination information 312.

Information 300 may be obtained via a sensor coupled to or in proximity to a patient or may be obtained via other sources. Various examples of a sensor are later described in association with at least FIGS. 11-12, 13A-13I, 14A-14B, 16A-16C, 17A-17B, and 21.

In some examples, one type of information may be derived from another type of information. For instance, via filtering or other processing mechanisms, at least some forms of cardiac information 304 (e.g. heart rate) may be determined or derived from respiratory information 302, where the respiratory information 302 is determined via a sensor. By looking at the behavior (e.g. increasing, decreasing, stable, high variability, low variability, high disorganization, low disorganization, etc.) of this derived/determined heart rate information alone and/or with other factors, one may determine a cardiac condition.

In some examples, respiratory information 302 is gathered during daytime (e.g. non-sleep) activities to detect the potential presence or worsening of non-cardiac diseases, such as but not limited to, pulmonary diseases (in addition to the particular pulmonary issues directly associated with sleep disordered breathing). In some examples, other information 310 may gather information and/or determine information regarding such non-cardiac physiologic conditions and/or diseases. In one example, such other information 310 includes pulmonary information. In some examples, such pulmonary information includes pulmonary disease information, such as but not limited to, chronic obstructive pulmonary disease (COPD), exacerbation of chronic obstructive pulmonary disease (ECOPD), etc.

In some examples, a change in respiratory information 302 may be indicative of future changes in cardiac information 304, sleep quality information 306, and sleep disordered breathing (SDB) information 308. In some examples, a change in respiratory information 302 may be indicative of future changes in other information 310, such as pulmonary disease information. For instance, for a patient already known to have chronic obstructive pulmonary disease (COPD), an increase in a patient's respiratory rate (e.g. one type of respiratory information 302) and/or reduced tidal volume may signal a forthcoming exacerbation of chronic obstructive pulmonary disease (ECOPD). Accordingly, in some examples, a therapy device and/or monitoring resource (70, 60 in FIG. 1) may be programmed to store a known non-cardiac disease information in other information 310 along with an association with another type of information, such as respiratory information 302, such that when the therapy device and/or monitoring resource detects a change in respiratory information 302 (e.g. increase in respiratory rate 302), the therapy device and/or monitoring resource automatically provides a notification for a clinician/patient that evaluation and/or intervention of the patient may be warranted regarding their pulmonary disease state in order to prevent or mitigate the pulmonary disease, such as preventing or mitigating ECOPD.

Accordingly, in such examples, the therapy device and/or manager may be programmed regarding various disease states of the patient to enable the therapy device and/or monitoring resource to act as an early warning system for non-cardiac conditions and/or non-OSA conditions upon detection of a change in respiratory information 302 or other types of information 300 monitored (e.g. gathered, determined, etc.) via a therapy device and/or monitoring resource.

In some examples, information 300, including other information 310, may be uploaded from an external source into a therapy device and/or manager. With further reference to FIG. 9, in some examples, sleep disordered breathing (SDB) information 308 is derived or determined from cardiac information 304. For instance, one example may comprise performing apnea detection from an electrocardiogram signal and/or other signals sensing cardiac activity.

In some examples, sensor 344 for obtaining information 300 (FIG. 9) may form part of a therapy device 340, as shown in FIG. 10A according to one example of the present disclosure. Therapy device 340 also comprises stimulator circuitry 342, which may take the forms described in association with at least FIGS. 6A, 7, 10A-10B, 19.

However, in some examples, sensor 354 for obtaining information 300 (FIG. 9) may be separate from, and independent of a therapy device 350 as shown in FIG. 10B, according to one example of the present disclosure. Like therapy device 340 in FIG. 10A, therapy device 350 in FIG. 10B also comprises stimulator circuitry 342. In some examples, while separate from and independent of therapy device 350, sensor 354 is dedicated to providing sensed information to therapy device 350. In some examples, sensor 354 is not dedicated to providing sensed information to therapy device 350. As such, sensor 354 may be part of a system which is independent of therapy device 350 or sensor 354 may be a standalone sensor not associated with any other system or device.

FIG. 11 is a block diagram schematically representing sensors 370, according to one example of the present disclosure. In some examples, sensors 370 may correspond to the sensors (e.g., 344 in FIG. 10A; 354 in FIG. 10B; 400 in FIG. 12; 654 in FIG. 16A; 702 in FIG. 17A; 712 in FIG. 17B; and 769 in FIG. 21) as previously described or later described in the examples of the present disclosure.

In some examples, sensor 370 is an implantable sensor 372 which is couplable relative to a patient's body via being implanted within a patient's body. Via such implantation, the sensor 372 is at least coupled mechanically relative to the patient's body. Moreover, via such implantation, the sensor 372 is further coupled relative to the patient's body according to the particular sensor modality (e.g. FIG. 12) of the implantable sensor 372, which may be electrical (e.g. impedance, etc.), mechanical (e.g. pressure, motion, etc.), chemical, etc.

In some examples, implantable sensor 372 forms part of another component implanted within the patient's body, such as a pulse generator (e.g. pulse generator 200 in FIG. 6C). In such examples, the sensor 372 may form part of the housing of the pulse generator and therefore may be exposed to the internal environment of the patient. On the other hand, in such examples, the sensor 370 may be housed internally within the pulse generator and be isolated from the internal environment of the patient. While a fuller discussion of sensor types 400 is reserved until a later discussion of FIG. 12, it will be noted that an accelerometer (e.g. 406 in FIG. 12) is one example of an implantable sensor, which is internally housed within a pulse generator.

In some examples, implantable sensor 372 may comprise stand-alone implantable sensors distributed throughout the patient's body and which communicate wirelessly to a SDB therapy device or to an external device that integrates the sensed data. For instance, one stand-alone implantable sensor may comprise an oxygen sensor.

In some examples, sensor 370 comprises an external sensor 374 that remains external to a patient's body. The external sensor 374 may be a wearable sensor 380, and therefore may at least releasably couplable relative to the patient's body. In some examples, the external sensor 374 comprises an environment sensor 382, which is present in and/or part of the patient's environment 382 and which senses information from the patient and/or regarding the environment in which the patient is present. However, in some instances, the environment sensor 382 is not couplable relative to the patient's body while in other instances, the environment sensor 382 is couplable relative the patient's body.

In some examples, a wearable sensor 380 may be used to sense physiologic information (such as heart rate variability) such that the wearable sensor 380 need not be part of an implantable therapy device or external therapy device. Rather, one may simply add the wearable sensor 380 at a later time to monitor cardiac parameters in association with a therapy performed to alleviate sleep disordered breathing.

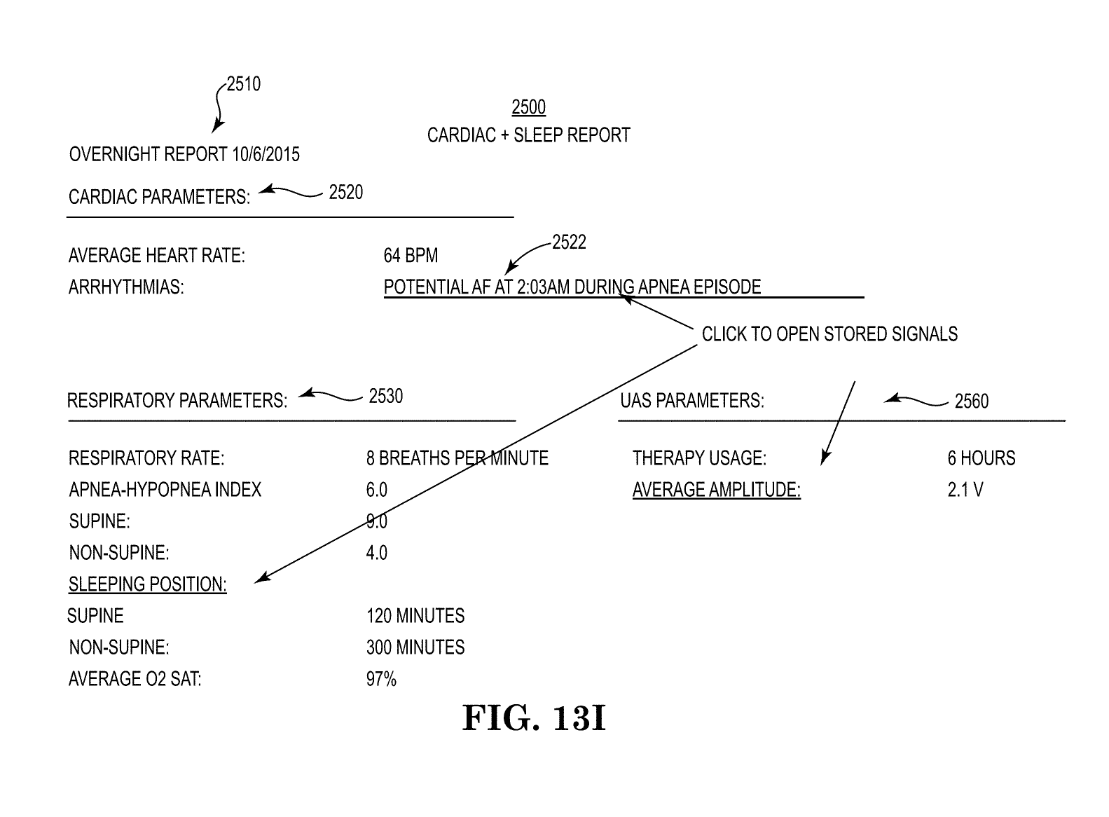

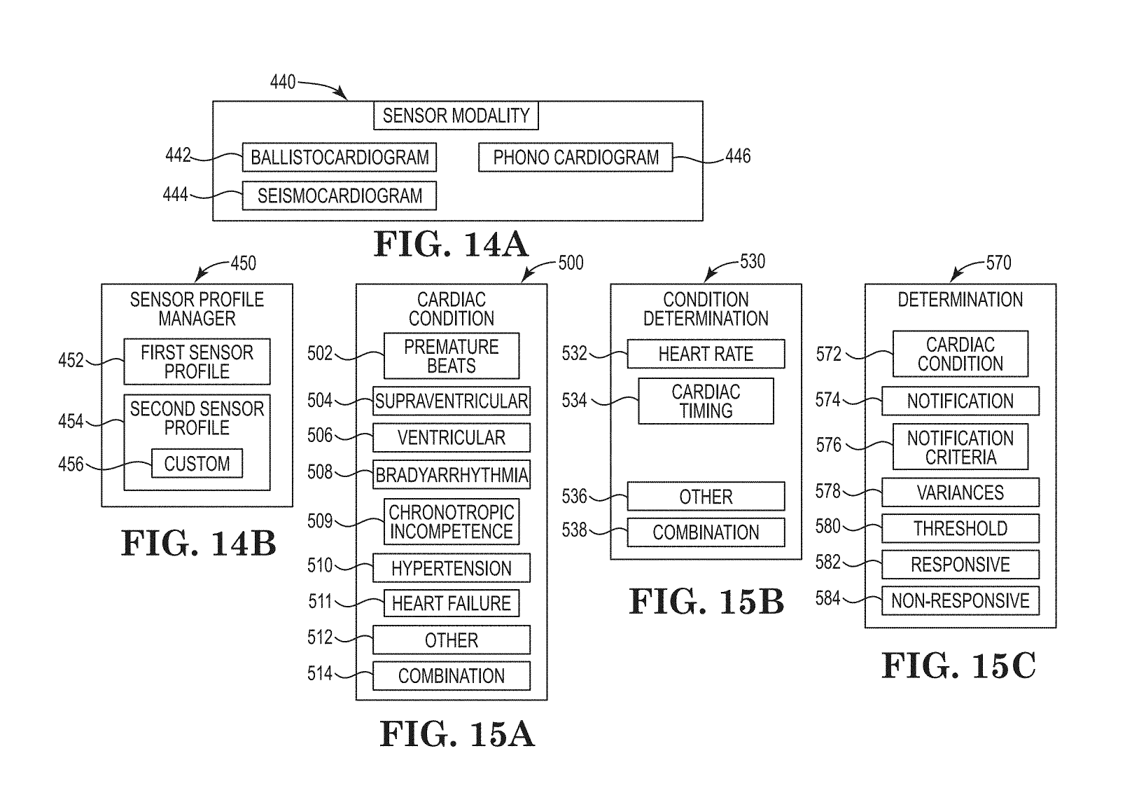

In some examples, a wearable sensor 380 may comprise a commercially available wearable sensor which includes an array of sensors for measuring heart rate (e.g. LED, optical sensor), sleep quality/motion (e.g. 3D accelerometer), ambient light, In some instances, the wearable sensor 380 includes a touchscreen display to facilitate monitoring the sensed conditions. In some instances, the wearable sensor 380 includes a wireless communication tool for communicating with a dongle, mobile device, etc. via a wireless communication protocol (e.g. Bluetooth, NFC, etc.). In one instance, such a wearable sensor 380 is available from FitBit, Inc. of San Francisco, Calif. In some examples, such a system may include a single sensor or array of sensors which provide respiratory information 302, cardiac information 304, sleep quality information 306, sleep disordered breathing (SDB) information 308, and/or other information 310 (FIG. 9). In some examples, this information may be coordinated with information sensed or determined via a sleep disordered breathing therapy device. For instance, in some examples, such wearable sensor arrangements cooperate with a sensor profile manager 450, as later further described in association with at least FIG. 14B.

In some examples, external sensor(s) 374 may be used to measure parameters, such as blood pressure, weight, etc. which may be used to identify a drug-resistant hypertension and any potential correlation or link between sleep disordered breathing (e.g. obstructive sleep apnea) and drug-resistant hypertension.

In some examples, information from external sensors 374 can be coordinated with information from implantable sensors 372. For instance, information from external sensors 374 or other external information sources, such as weather/environmental reports, can be coordinated with information from implanted sensors 372 to provide guidance to asthmatic patients on whether it's safe to go outside based on previous respiratory/weather correlations and situations.

In some examples, external sensor 374 comprises an integrated external sensing system for monitoring sleep quality, heart rate, breathing rhythm, movement, sleep stages, snoring, and sleep environment (e.g., noise level and light). One example system comprises the Beddit® system available from www.beddit.com. In some examples, such a system may provide respiratory information 302, cardiac information 304, sleep quality information 306, sleep disordered breathing (SDB) information 308, and/or other information 310 (FIG. 9). In some examples, this information may be coordinated with information sensed or determined via a sleep disordered breathing therapy device. For instance, in some examples, such external sensor arrangements cooperate with a sensor profile manager 450, as later further described in association with at least FIG. 14B.

In some examples, an external sensor(s) 374 may comprise clinically available diagnostic equipment such as ECG sensors, a blood pressure cuff, oxygen sensor, etc.

In some examples, external sensor(s) 374 may be incorporated into a patient remote, such as one of the access tools 131-133. In some examples, external sensor(s) 374 can measure parameters associated with an apnea-hypopnea index (AHI). In such examples, the external sensor 374 can sense pulse transit times, which vary during respiration.

In some instances, the environment sensor 382 shown in FIG. 11 comprises a non-contact sensor 384, which does not make contact with the patient. Accordingly, in such instances, the non-contact sensor 384 is at least not mechanically couplable relative to the patient's body. However, in some examples, the non-contact sensor 384 may be couplable relative to the patient's body in at least the sense that the particular sensor modality can relate to the patient's body in at least some fashion to obtain physiologic information regarding the patient.

For instance, at least some types of a non-contact sensor 384 are later described more fully regarding sensor type 400 in association with at least FIG. 12. In one instance, non-contact sensor 384 comprises at least some of substantially the same features and attributes as the non-contact sensor paradigm described in Heneghan et al. U.S. Pat. No. 5,562,526, which may provide respiratory information 302, cardiac information 304, sleep quality information 306, sleep disordered breathing (SDB) information 306, and/or other information. In one instance, one such system is available from Resmed Corporation of San Diego, Calif.

In some instances, the non-contact sensor 384 incorporates or cooperates with one of the sensor modalities described in association with at least FIG. 12, such as but not limited to, a radiofrequency sensor 408. The signal produced by sensing via the radiofrequency sensor 408 (also a non-contact sensor 384) may be processed to detect patient motion/activity, breathing (e.g. respiratory rate), heart rate, and/or a sleep stage of the patient. In some instances, physiologic information, such as cardiac information, detected via the radiofrequency sensor 408 (a non-contact sensor 384) may take the form of a ballistocardiogram or seismocardiogram, which are both further described later in association with at least FIG. 14A. Among other attributes, in at least some examples the ballistocardiogram and/or seismocardiogram may obtain at least cardiac information without contacting the patient and thus may sometimes be referred to as unobtrusive cardiac sensing.

In some examples, sensor 370 may comprise a sensor providing a combination sensor 376, which combines at least some aspects of the various implantable sensor 372 and external sensor 374.

FIG. 12 is a block diagram schematically representing a sensor type 400 according to one example of the present disclosure. In some examples, sensor type 400 corresponds to a sensor (e.g., 344 in FIG. 10A; 354 in FIG. 10B; 370 in FIG. 11; 654 in FIG. 16A; 702 in FIG. 17A; 712 in FIG. 17B; 769 in FIG. 21) as previously described or later described in the examples of the present disclosure.

As shown in FIG. 12, sensor type 400 comprises various types of sensor modalities 402-422, any one of which may be used for determining, obtaining, and/or monitoring respiratory information 302, cardiac information 304 (e.g. positive cardiac conditions and/or negative cardiac conditions), sleep quality information 306, sleep disordered breathing-related information 308, and/or other information 310 as previously described in association with at least FIG. 9.

As shown in FIG. 12, in some examples sensor type 400 comprises the modalities of pressure 402, impedance 404, accelerometer 406, airflow 407, radiofrequency (RF) 408, optical 410, electromyography (EMG) 412, electrocardiography (ECG) 414, ultrasonic 416, acoustic 418, image 419, and/or other 420. In some examples, sensor type 400 comprises a combination 422 of at least some of the various sensor modalities 402-420.

It will be understood that, depending upon the attribute being sensed, in some instances a given sensor modality identified within FIG. 12 may include multiple sensing components while in some instances, a given sensor modality may include a single sensing component. Moreover, in some instances, a given sensor modality identified within FIG. 12 may include monitoring circuitry and/or communication circuitry. However, in some instances a given sensor modality in FIG. 12 may omit such monitoring and/or communication circuitry but may cooperate with such monitoring or communication circuitry located elsewhere.

In some examples, a pressure sensor 402 may sense pressure associated with respiration and can be implemented as an external sensor 374 (FIG. 11) and/or an implantable sensor 372 (FIG. 11). In some instances, such pressures may include an extrapleural pressure, intrapleural pressures, etc. For example, one pressure sensor 402 may comprise an implantable respiratory sensor, such as that disclosed in Ni et al. U.S. Patent Publication 2011-0152706, published on Jun. 23, 2011, titled METHOD AND APPARATUS FOR SENSING RESPIRATORY PRESSURE IN AN IMPLANTABLE STIMULATION SYSTEM.

In some instances, pressure sensor 402 may include a respiratory pressure belt worn about the patient's body.

In some examples, a pressure sensor 402 can sense sound and/or pressure waves at a different frequency than occur for respiration (e.g. inspiration, exhalation, etc.). In some instances, this data can be used to monitor cardiac parameters of patients via a respiratory rate and/or a heart rate. In some instances, such data can be used to approximate electrocardiogram information, such as a QRS complex. In some instances, the detected heart rate is used to identify a relative degree of organized heart rate variability, in which organized heart rate variability may enable detecting apneas or other sleep disordered breathing events, which may enable evaluating efficacy of sleep disordered breathing. In some instances, the detected heart rate is used to identify disorganized heart rate variability, which may enable detecting cardiac disorders, such as arrhythmias (e.g. atrial fibrillation, ventricular tachycardia, etc.), for which cardiac intervention (e.g. ablation, drug therapy, etc.) may be appropriate.

In some examples, pressure sensor 402 comprises an implantable blood pressure sensor which is separate from a therapy device and which may be used to monitor cardiac parameters.

In some examples, pressure sensor 402 is locatable in close proximity to the patient's heart to optimize detection of cardiac information 304.

In some examples, pressure sensor 402 comprises an intracardiac absolute pressure sensor. In some instances, this pressure sensor is used to detect respiration and/or arterial pressure. This pressure sensor also may involve a training mode in which field calibration is applied via use of an external sensor (wearable atmospheric blood pressure), thereby ensuring accuracy of the intracardiac absolute pressure sensor. Due to component sensitivity, manufacturing variability, implant variability, and/or system interactions, in at least some instances, it may be more accurate and simpler to perform a field calibration (such as but not limited to the above-described field calibration) with the sensor in its final functional state rather than trying to calibrate the sensor to an absolute scale at the component level in the manufacturing environment. In this way, an implantable pressure sensor in accordance with at least some examples of the present disclosure may be utilized with simpler manufacturing processes than if a pre-calibrated sensor were implanted.

In some examples, use of the pressure sensor 402 is paired with obtaining a far field ECG, in which the ECG signal is used to filter out or blank out cardiac artifacts from the pressure sensor signal.

In some examples, the pressure sensor 402 is used to determine minute ventilation. Among other benefits, the determined minute ventilation may be used to make long term evaluations regarding pulmonary disease.

As shown in FIG. 12, in some examples one sensor modality includes air flow sensor 407, which can be used to sense respiratory information 302, sleep disordered breathing-related information 308, sleep quality information 306, etc. In some instances, air flow sensor 407 detects a rate or volume of upper respiratory air flow.

As shown in FIG. 12, in some examples one sensor modality includes impedance sensor 404, which may be implemented in some examples via various sensors distributed about the upper body for measuring a bio-impedance signal, whether the sensors are internal and/or external. In some instances, the sensors are positioned about a chest region to measure a trans-thoracic bio-impedance. In some instances, at least one sensor involved in measuring bio-impedance can form part of a pulse generator, whether implantable or external. In some instances, at least one sensor involved in measuring bio-impedance can form part of a stimulation element and/or stimulation circuitry. In some instances, at least one sensor forms part of a lead extending between a pulse generator and a stimulation element.

In some examples, impedance sensor 404 may take the form of electrical components not used in a SDB therapy device. For instance, some patients may already have a cardiac therapy device (e.g. pacemaker, defibrillator, etc.) implanted within their bodies, and therefore have some cardiac leads implanted within their body. Accordingly, the cardiac leads may function together or in cooperation with other resistive/electrical elements to provide impedance sensing.

In some examples, whether internal and/or external, impedance sensor(s) 404 may be used to sense an electrocardiogram (ECG) signal.

As shown in FIG. 12, in some examples one sensor modality includes an accelerometer 406. In some instances, accelerometer 406 is generally incorporated within or associated with device 171, 210 or may be incorporated within or form part of a pulse generator (e.g. 200 in FIG. 6C). For instance, in some examples of a pulse generator, a housing (e.g. can) contains numerous components such as control circuitry, stimulation, and also may contain an accelerometer 406 within the housing. However, in some examples, the accelerometer 406 may be separate from, and independent of, the pulse generator (e.g. 200 in FIG. 6C). In some examples, accelerometer 406 can enable sensing body position, body posture, and/or body activity regarding the patient, which may be indicative of behaviors from which sleep quality information 306 or sleep disordered breathing (SDB) information 308 may be determined. For instance, sleep position (e.g. left side, right side, supine, etc.) may be used to determine the effectiveness of SDB therapy according to sleep position, and in some instances, the SDB therapy may be automatically adjusted based on the orientation (i.e. sleep position) of the patient. In some instances, this information regarding sleep position may be communicated to the patient during a sleep period in order to induce the patient to change their sleep position into one more conducive to efficacious SDB therapy. In some examples, the communication may occur by an audible or vibratory alarm implemented via wireless communication to a patient remote or via direct muscle stimulation via wireless communication to a wearable muscle stimulation device.

FIG. 13A is a diagram 2000 schematically representing some aspects of accelerometer sensing in association with some aspects of sleep quality, according to one example of the present disclosure. As shown in FIG. 13A, diagram 2000 juxtaposes several different types of information/waveforms, such as a snoring intensity waveform 2010, a respiratory waveform 2020, a stimulation profile 2025, a sleep position profile 2030, and a sleep apnea index waveform (e.g. AHI) 2040. In some examples, the sleep apnea index waveform provides at least one measure of sleep quality among several potential measures of sleep quality.

In one aspect, the information shown in diagram 2000 corresponds to information obtained via automatic storage of at least minute-by-minute sleep data, therapy data, positional data, etc.

In some examples, at least one accelerometer 406 can be used to obtain the snoring intensity waveform 2010, respiratory waveform 2020, and/or sleep position profile 2030. In some examples, other sensing elements are used to obtain such information as described within at least some examples throughout the present disclosure.

In some examples, the snoring intensity waveform 2010 includes a first portion 2011 having a first generally constant value and a second portion 2012 having a second value generally higher than the first value. In some examples, the respiratory waveform 2020 includes a first portion (e.g. series of respiratory cycles) of generally normal respiration followed by a second portion 2022 of irregular respiratory cycles 2023, 2024, 2029, etc. Accordingly, the increased snoring intensity generally coincides with the second portion 2022 representing irregular breathing.

In some examples, stimulation profile 2025 includes a series of stimulation pulses at a particular intensity (e.g. 2.1 V) with some stimulation pulses 2026 being of longer duration and less frequency and some stimulation pulses 2027 of shorter duration and higher frequency. In one aspect, the shorter, more frequent stimulation pulses are applied during the irregular respiratory cycles 2023, 2024, 2029.

In some examples, sleep position profile 2030 includes a first sleep position 2032 (e.g. left side) and a second sleep position 2034 (e.g. supine). It can be observed that the second sleep position 2034 generally coincides with the elevated snoring intensity 2012 and irregular respiratory cycles 2023, 2024, 2029.

In some examples, sleep apnea index waveform 2040 includes a first portion 2042 having a generally constant value and a second portion 2044 in which the index (e.g. AHI) increases over time. It can be observed that the supine sleep position 2034 generally coincides with the elevated snoring intensity 2012, irregular respiratory cycles 2023, 2024, 2029, and supine sleep position 2034.

Among other uses, the information in diagram 2000 may be employed by a clinician to adjust stimulation therapy and/or employed by a therapy device (and/or manager) to automatically adjust stimulation therapy to cause a decrease in the moving average of the sleep apnea index (e.g. AHI) represented by waveform 2040. Moreover, as previously mentioned this information may be used to communicate to the patient via audio or non-audio techniques to change their sleep position to a position (e.g. left side) more amenable to regular respiration (e.g. portion 2021).

In some examples, some portions schematically represented in FIGS. 13A-13I may function as (or correspond to) at least some instances of a user interface for cardiac-related monitoring according to at least some examples of the present disclosure, whether the user interface is a clinician user interface or a patient user interface. It will be understood that in some examples, user interface portions represented in one Figure within FIGS. 13A-13I may be combined in a complementary manner with user interface portions in another one of FIGS. 13A-13I or with user interface portions among several figures within FIGS. 13A-13I and/or FIGS. 4A-5B.

FIG. 13B is a diagram 2050 schematically representing some aspects of accelerometer sensing in association with some aspects of sleep quality, according to one example of the present disclosure. In general terms, diagram 2050 maps several waveforms over an entire night of sleep. Among other things, relative to a general timeline 2055, FIG. 13B provides a juxtaposition of a sleep apnea index waveform 2060, a stimulation profile 2070, and a sleep position profile 2080. As can be seen via FIG. 13B, in some examples a supine sleep position (2082) results in increases in amplitude (2062) of the apnea index (e.g. AHI), and which is generally matched via a therapy device with an increase in the intensity (e.g. amplitude) of stimulation (2072). However, upon the patient switching to a side sleep position (e.g. left side) 2084, the apnea index decreases (2064), thereby resulting in the therapy device reducing stimulation intensity (2074). It will be understood that in some examples stimulation intensity may be adjusted via other parameters, such as pulse width, frequency, etc. in combination with or separate from amplitude adjustments.

In some examples, accelerometer 406 enables acoustic detection of cardiac information 304, such as heart rate and/or electrocardiogram (ECG) waveforms, including QRS complexes. In some examples, measuring the heart rate includes sensing heart rate variability. In some examples, accelerometer 406 can sense respiratory information, such as but not limited to, a respiratory rate. In some examples, whether sensed via an accelerometer 406 alone or in conjunction with other sensors, one can monitor cardiac information 304 and respiratory information 302 simultaneously by exploiting the behavior of ECG signal in which an ECG waveform can vary with respiration.

FIG. 13C is a diagram 2200 schematically representing some aspects of acoustic sensing of cardiac information and respiratory information, according to one example of the present disclosure. In some examples, the acoustic sensing demonstrated in FIG. 13C is performed via accelerometer 406. Among other things, the accelerometer 406 can enable various forms of cardiac timing measurements, such as but not limited to, heart rate detection, QT timing detection, etc. This cardiac timing, in turn, enables heart rate variability measurements.

As shown in diagram 2200, accelerometer 406 produces a raw output waveform 2210, which is split (2212) via filtering with a high pass filter 2220 to produce a phonocardiogram waveform 2222 and via filtering with a low pass filter 2230 to produce a respiratory waveform 2232. Among other features, the phonocardiogram waveform 2222 includes an S1 component, which correlates with a QRS complex in an ECG waveform, and a S2 component, which correlates with a T-wave component in an ECG waveform 2224. Accordingly, via this arrangement the accelerometer 406 may sense both cardiac motion and respiratory motion, which may be differentiated and identified via application of the respective different frequency filters 2220 and 2230. In one aspect, as shown in FIG. 13D, a Wiggers diagram 2250 illustrates (among other things), portions of the phonocardiogram which coincide with or correspond with portions of an electrocardiogram (ECG). In one aspect, this Wiggers Diagram may be obtained at https://commons.wikimedia.org/wiki/File:Wiggers Diagram.svg#filelinks.

In some examples, accelerometer 406 enables detection of sleep/awake via the sensing of motion, position, posture and/or activity of the patient, along with other parameters determinable via the accelerometer 406. In some instance, this information may be used to implement automatic control of SDB therapy to enhance therapeutic efficacy.

In some examples, the accelerometer 406 comprises an external sensor 374. In some instances, when embodied as an external sensor, the accelerometer 406 may comprise a wearable sensor, such as an accelerometer incorporated into a band or belt worn about a portion of the body (e.g. wrist, chest, arm, leg, torso, etc.).

In some examples, the accelerometer 406 may be used to detect sleep disordered breathing events during the sleep period and may be used continuously to detect arrhythmias.

In some examples, radiofrequency sensor 408 shown in FIG. 12 enables non-contact sensing of various physiologic parameters and information, such as but not limited to cardiac information 304, respiratory information 302, motion/activity, and/or sleep quality, such as previously described regarding non-contact sensor 384 in association with at least FIG. 11. In some examples, radiofrequency sensor 408 enables non-contact sensing of other physiologic information.

Accordingly, FIG. 13E is a diagram 2400 schematically representing RF-based non-contact sensing of respiratory information, according to one example of the present disclosure. As shown in diagram 2400 of FIG. 13E, a sensing arrangement 2410 includes a radio-frequency (RF) sensor 2412 which determines chest motion based on Doppler principles 2420 via signals sent and received by sensor 2412 relative to the chest of the patient 2414. The sensor 2412 can be located anywhere within the vicinity of the patient 2414, such as various locations within the room (e.g. bedroom) in which the patient is sleeping. In some examples, the sensor 2412 is coupled to a non-dedicated mobile device 132 (e.g. mobile phone in one example) or other access tool in array 130 (FIG. 3B) to enable data transmission relative to other components of a therapy device and storage in such other components. In some examples, sensing arrangement 2410 comprises at least some of substantially the same features and attributes as non-contact sensor 384, as previously described in association with FIG. 11.

In some examples, one sensor modality may comprise an optical sensor 410 as shown in FIG. 12. In some instances, optical sensor 410 may be an implantable sensor 372 and/or external sensor 374 (FIG. 11). For instance, one implementation of an optical sensor 410 comprises an external optical sensor for sensing heart rate and/or oxygen saturation via pulse oximetry. In some instances, the optical sensor 410 enables measuring oxygen desaturation index (ODI). In some examples, the optical sensor 410 comprises an external sensor removably couplable on the finger of the patient.

In some examples, optical sensor 410 can be used to measure ambient light in the patient's sleep environment, thereby enabling an evaluation of the effectiveness of the patient's sleep hygiene and/or sleeping patterns.

As shown in FIG. 12, in some examples one sensor modality comprises EMG sensor 412, which records and evaluates electrical activity produced by muscles, whether the muscles are activated electrically or neurologically. In some instances, the EMG sensor 412 is used to sense respiratory information 302, such as but not limited to, respiratory rate, apnea events, hypopnea events, whether the apnea is obstructive or central in origin, etc. For instance, central apneas may show no respiratory EMG effort.

In some instances, the EMG sensor 412 may comprise a surface EMG sensor while, in some instances, the EMG sensor 412 may comprise an intramuscular sensor. In some instances, at least a portion of the EMG sensor 412 is implantable within the patient's body and therefore remains available for performing electromyography on a long term basis.

In some examples, one sensor modality may comprise ECG sensor 414 which produces an electrocardiogram (ECG) signal. In some instances, the ECG sensor 414 comprises a plurality of electrodes distributable about a chest region of the patient and from which the ECG signal is obtainable. In some instances, a dedicated ECG sensor(s) 414 is not employed, but other sensors such as an array of bio-impedance sensors 404 are employed to obtain an ECG signal. In some instances, a dedicated ECG sensor(s) is not employed but ECG information is derived from a respiratory waveform, which may be obtained via any one or several of the sensor modalities in sensor type 400 in FIG. 12. In some examples, ECG sensor 414 is embodied as an accelerometer 406 as previously described in association with FIG. 12 and/or in association with at least FIG. 13A-13D.

In some examples, an ECG signal obtained via ECG sensor 414 may be combined with respiratory sensing (via pressure sensor 402 or impedance sensor 404) to determine minute ventilation, as well as a rate and phase of respiration.

In some examples, the ECG signal obtained via ECG sensor 414 may be combined with cardiac output sensing (via pressure sensor 402 or impedance sensor 404). In one aspect, the cardiac output is the product of heart rate times stroke volume. In one aspect, a higher pressure of left ventricle (LV) contractility (as represented by dP/dt) may enable inferring higher cardiac output, and therefore the left ventricle (LV) contractility may provide a relative measure of cardiac stroke volume. In some examples, this arrangement may be implemented via placing the ECG sensor 414 in the aorta or in the left ventricle. In some examples, the cardiac output sensing enables determining arterial pulse pressure (difference between systolic and diastolic pressure readings) because the stroke volume may be proportional to the arterial pulse pressure.

In some examples, the ECG sensor 414 may be exploited to obtain respiratory information (e.g. at least 302 in FIG. 9). FIG. 13F is a diagram schematically representing derivation of respiratory information from a cardiac waveform, according to one example of the present disclosure. In one aspect, diagram 2300 in FIG. 13F provides a juxtaposition of cardiac timing information and respiratory information. As shown in FIG. 13F, diagram 2300 includes a raw electrocardiogram waveform 2310, which is filtered via a high pass filter 2220 to obtain a conditioned electrocardiogram waveform 2324 and filtered via a low pass filter 2230 to obtain a respiratory waveform 2332. Accordingly, both respiratory information 302 and cardiac information 304 (FIG. 9) can be obtained via an ECG sensor 414. In some examples, as noted elsewhere, ECG sensor 414 may be implemented, at least in part, as an accelerometer 406 (FIG. 12).

FIG. 13G is a diagram 2350, according to one example of the present disclosure, further illustrating aspects of a respiratory waveform derived from an ECG waveform (e.g. ECG sensor 414), such as described in association with FIG. 13F. Accordingly, diagram 2350 juxtaposes a normal ECG 2324 and respiratory waveform 2332 as in FIG. 13F, except further juxtaposing a RR interval profile 2360 with the other waveforms 2324, 2332. In one aspect, diagram 2350 demonstrates how aspects of cardiac timing, such as R-R intervals and/or P-R intervals, vary with respiration. For instance, one can observe how the R-R interval waveform 2360 increases and decreases in a pattern which generally corresponds to inspiration and exhalation, respectively. Among other uses, this information may enable identifying correlations, relationships, and/or associations between cardiac disorder parameters, cardiac health parameters, sleep parameters, and/or respiratory parameters.

As shown in FIG. 12, in some examples one sensor modality includes an ultrasonic sensor 416. In some instances, ultrasonic sensor 416 is locatable in close proximity to an opening (e.g. nose, mouth) of the patient's upper airway and via ultrasonic signal detection and processing, may sense exhaled air to enable determining at least respiratory information 302, sleep quality information 306, sleep disordered breathing information 308, and/or other information 310. In some instances, ultrasonic sensor 416 may comprise at least some of substantially the same features and attributes as described in association with at least Arlotto et al. PCT Published Patent Application 2015-014915 published on Feb. 5, 2015.

In some examples, an acoustic sensor 418 shown in FIG. 12 may be employed to sense respiratory information 302 (e.g. breathing rate, respiratory waveform, etc.), cardiac information 304 (e.g. heart rate, cardiac waveform, etc.), sleep quality information 306, sleep disordered breathing (SDB) information 308, and/or other information 310, as shown in FIG. 9. In some examples, an acoustic sensor 418 can implement sonar detection schemes via mobile device 131, 132 (FIG. 3B) to obtain at least respiratory information 302. For instance, the acoustic sensor 418 be part of and/or cooperate with a smartphone running an application (i.e. mobile app) designed to monitor apnea events thru sonar, such as described in “Contactless Sleep Apnea Detection on Smartphones” reported by the University of Washington in May 2015 at The 13th International Conference on Mobile Systems, Applications, and Services in Florence, Italy.

In some examples, other sensor 420 comprises any other type of sensor or sensor modality useful for sensing and monitoring respiratory information 302, cardiac information 304, sleep quality information 306, sleep disordered breathing information 308, and/or other information 310 (FIG. 9). For instance, in some examples an “other” sensor 420 may comprise a temperature sensor for sensing the ambient temperature in the patient's sleep environment and/or a temperature of the patient before, during, and after sleep, as such temperatures may affect sleep quality or may reflect information about a respiratory condition, cardiac condition, or sleep disordered breathing.

FIG. 13H is a diagram 2450 schematically representing a juxtaposition of respiratory information, cardiac information, and sleep information, according to one example of the present disclosure. In general terms, diagram 2450 can facilitate identifying a period of atrial arrhythmia potentially due to apnea based on factors, such as observation of an elevated heart rate, the atrial rate being greater than the ventricular rate, etc., both of which generally coincide with an apneic period.

In some examples, diagram 2450 is displayable as part of a clinician user interface, such as interface 1000 (FIG. 4A). In some examples, diagram 2450 includes a respiratory waveform 2020 and stimulation profile 2025 like that in FIG. 13A, as well as a heart rate profile 2460, a V-A association waveform 2470, and a sleep position profile 2030 like that in FIG. 13A. As shown in FIGS. 13A and 13H, the heart rate profile 2460 includes a first portion 2462 and a second portion 2463. The first portion 2462 represents a baseline heart rate while the second portion 2463 represents heart rate variability. In the example shown in FIG. 13A, the second portion 2463 includes peaks 2464, 2466 (e.g. elevated heart rate) and valley 2467.

As further shown in FIG. 13H, the V-A association waveform 2470 includes a first baseline portion 2472 and a second portion 2473 exhibiting variability occurring in synch with the respiratory irregularity (i.e. irregular breathing) 2022. Meanwhile, the sleep position profile 2030 indicates that the respiratory irregularities 2023, 2024, 2029, elevated heart rate 2464, 2466, and increased values of the V-A association 2474, 2476 correspond to a supine sleep position 2034.

In one aspect, the V-A association waveform represents a ratio between the ventricular and atrial rate. This ratio is normally 1:1, and any deviation of 1:n (n>1) indicates an atrial arrhythmia, or n:1 (n>1) indicating a ventricular arrhythmia.

As shown in FIG. 13H, a strong correlation is present between peaks of the V-A association 2474, 2476, an elevated heart rate 2464, 2466, and irregular respiratory cycles (e.g. 2023, 2029).

FIG. 13I is a diagram 2500 schematically representing an overnight patient report 2510 including at least cardiac information, respiratory information, and sleep information, according to one example of the present disclosure. As shown in diagram 2500 of FIG. 13I, in some examples the overnight patient report 2510 includes cardiac parameter portion 2520, respiratory parameter portion 2530, and Upper Airway Stimulation therapy parameter portion 2560.

In some examples, the cardiac parameter portion 2520 displays information regarding an average heart rate and any arrhythmias, such as a potential instance of atrial fibrillation (AF) 2522 during an apnea episode at a particular time. In some examples, the respiratory parameter portion 2530 monitors values of various measured respiratory parameters, such as but not limited to, respiratory rate, apnea index (e.g. AHI) in supine and non-supine positions, sleeping position durations, and oxygen saturation.

In some examples, therapy parameter portion 2560 includes a total duration of therapy for that night and an average amplitude of stimulation.

In some examples, diagram 2500 is displayable and interactively engageable as a user interface (e.g. 140 in FIG. 3C). For instance, in some examples certain parameters, such as sleep position (within respiratory parameter portion 2530) are implemented at hot links, such that engagement of the link causes a graph of a stored signal (e.g. sleep position profile 2030 in FIG. 13H) to appear on the display exhibiting the diagram 2500. In some examples, a stimulation profile 2025 (FIG. 13H) is displayable in diagram 2500 upon “clicking” on the average amplitude parameter.

In some examples, diagram 2500 can be displayed and engaged as part of a clinician user interface 1000 (FIG. 4A) while in some examples, diagram 2500 can be displayed and engaged as part of a patient user interface (FIGS. 5A-5B). Moreover, as noted elsewhere, portions of diagram 2500 as a user interface can be combined in various combinations with user interface portions represented in at least FIGS. 4A-5B and/or FIGS. 13A-13H.

FIG. 14A is a block diagram schematically representing a sensor modality array 440, according to one example of the present disclosure. In some examples, sensor modality array 440 provides additional modes of sensing in addition to those described in association with at least FIGS. 11-13I. In some instances, the modalities 442, 444, 446 complement and/or implement at least one of the types of sensors described in association with at least FIGS. 11-13I.

Via the different sensor modalities 442, 444, 446, at least cardiac information and/or respiratory information may be determined.

In some examples, one sensor modality 440 comprises a ballistocardiogram sensor 442 to determine at least cardiac-related information. In some instances, the ballistocardiogram sensor 442 may be implemented via at least accelerometer sensor 406, acoustic sensor 418, and/or radiofrequency sensor 408 in FIG. 12. In at least some instances, a ballistocardiogram may be understood as the measurement of the recoil forces of the body in reaction to cardiac ejection of blood into the vasculature.

In some examples, one sensor modality 440 comprises a seismocardiogram sensor 444 to determine at least cardiac-related information. In some instances, the seismocardiogram sensor 444 may be implemented via at least accelerometer sensor 406 acting in at least a vibratory/motion detecting mode. In some instances, the seismocardiogram sensor 444 may be implemented via a radiofrequency sensor 408. In at least some instances, a seismocardiogram may be understood as representing the local vibrations of the chest wall in response to the heartbeat.

In some examples, one sensor modality 440 comprises a phonocardiogram sensor 446. In some examples, a phonocardiogram sensor 446 may be implemented in a manner substantially similar as described in association with at least FIGS. 13C-13D.

FIG. 14B is a block diagram schematically representing a sensor profile manager 450, according to one example of the present disclosure. In some examples, sensor profile manager 450 forms part of and/or cooperates with therapy device and/or monitoring resource (110, 60 in FIG. 1). As shown in FIG. 14B, sensor profile manager 450 includes a first sensor profile function 452 and second sensor profile function 454. In some examples, the first sensor profile function 452 includes and/or monitors those sensors already associated with a therapy device and/or monitoring resource. Meanwhile, in some examples, the second sensor profile function 454 acts to receive sensor information from at least one commercially available sensor device or sensor array. The second sensor profile function 454 enables at least some of the sensors of the commercially available sensor device/array to supplement and/or replace sensors associated with the first sensor profile function 452.

In some examples, the second sensor profile function 454 includes an array of pre-programmed sensor profiles. In some example, each array of pre-programmed sensor profile corresponds to a different commercially available sensor device/array. For instance, one array can correspond to one wearable sensor array (e.g. 380 in FIG. 11) having at least some of substantially the same features and attributes as a wearable sensor array available under the trademark FitBit®. For instance, one array can correspond to one external sensor array (e.g. 374, 382 in FIG. 11) having at least some of substantially the same features and attributes as a sensor array available under the trademark Beddit®. In some examples, such commercially available sensor device/arrays may correspond to and/or include some features corresponding to one of the access tools 131-135 (FIG. 3B), such as non-dedicated mobile device 132.

In some examples, such commercially available sensor device/arrays can communicate securely with a therapy device (e.g. 70 in FIG. 1) and/or monitoring resource (e.g. 60 in FIGS. 1, 3A) to ensure reliable, safe operation of the therapy device and/or monitoring resource. In some examples, such secure communication is enabled and facilitated via one of the access tools 131-135, which establishes the secure communication channel. For instance, the commercially available sensor device/array may communicate directly with such “secure communication” device to establish a communication pathway between the commercially available sensor device/array and a therapy (e.g. 70 in FIG. 1) and/or monitoring resource (e.g. 60 in FIGS. 1, 3A). In some examples, the sensor profile manager 450 enables a therapy device and/or monitor to automatically recognize and implement a commercially available sensor device/array upon establishing a secure communication channel therebetween.

In some examples, the second sensor profile function 454 enables the therapy device and/or monitoring resource to seamlessly integrate and/or leverage the commercially available sensor device/arrays with the sensors associated with the first sensor profile function 452. The sensors associated with the first sensor profile function 452 may be on board sensors (e.g. accelerometer 406 on/in pulse generator (IPG)), implantable sensors, or external sensors in the manner described in association with at least FIGS. 9-12.

In some examples, second sensor profile function 454 is configured to integrate the use of sensors in access tools 131-135 (FIG. 3B) separately from a commercially available sensor device/array or in complementary association with a commercially available sensor device/array. In some examples, one such access tool in array 130 (FIG. 3B) comprises a non-dedicated mobile device 132, such as a smart phone, tablet, phablet, etc.

In some examples, second sensor profile function 454 includes a custom parameter 450 by which a custom sensor profile function can be built to receive sensor information from a customized sensor device/array.

In some examples, the sensor profile manager 450 can be updated to include changes to a sensor(s) in the first sensor profile function 452 and/or second sensor profile function 454. For instance, a sensor profile associated with a new commercially available sensor device/array can be uploaded to become part of the second sensor profile function 454.

FIG. 15A is a block diagram schematically representing cardiac condition array 500, according to one example of the present disclosure. As shown in FIG. 15A, in some examples, cardiac condition array 500 comprises premature beats condition 502, supraventricular condition 504, ventricular condition 506, bradyarrhythmia condition 508, chronotropic incompetence 509, hypertension 510, heart failure 511, and/or other condition 512. Meanwhile, combination condition 514 comprises a combination of at least two of the conditions 502-512 of array 500.

In some examples, any one of the conditions in array 500 may be sensed and/or monitored as cardiac information 304 (FIG. 9), via sensor 370 (FIG. 11), via one of the sensor modalities represented in the sensor type array 400 (FIG. 12), and/or other mechanisms available to a clinician.

In some examples, the supraventricular condition 504 includes, but is not limited to, atrial fibrillation, atrial flutter, and/or paroxysmal supraventricular tachycardia. In one sense, atrial fibrillation is associated with rapid, irregular, and/or unsynchronized contraction of the muscle fibers of the atrium of the patient's heart. In one sense, atrial fibrillation is identifiable by disorganized electrical impulses (sometimes originating in the roots of the pulmonary veins) overcoming the normal electrical pulses coming from the sinoatrial node. This phenomenon may lead to irregular conduction of impulses from the atria to the ventricles, such that the contraction and relaxation of the atria are out of synch with the ventricles of the heart.

In some examples, atrial fibrillation is recognizable via observing a standard deviation of Atrial-Atrial timings. In a normally functioning heart, the Atrial-to-Atrial timings are very tightly coupled. However, if one observes a large spread in Atrial to Atrial timings, this pattern may indicate atrial fibrillation. In one context, such as viewing a cardiac waveform (e.g. ECG), atrial fibrillation is associated with a large number of small P waves for a single QRS complex, such that the cardiac waveform exhibits a near absence of distinct P waves in the cardiac waveform.

In some examples, one can use the cardiac information 304 to observe a Ventricular-to-Atrial Beat Ratio, which is 1:1 in a normally function heart. However, if the V-A Beat Ratio is 1:n, wherein n>1 for a consistent period of time, then these values likely indicate atrial fibrillation.

In some examples, the ventricular condition 506 includes ventricular arrhythmias such as, but is not limited to, ventricular fibrillation and/or ventricular tachycardia. In some examples, if the above-referenced V-A Beat Ratio is 1:n, wherein n<1, this Beat Ratio may indicate a ventricular arrhythmia. In some examples, ventricular arrhythmia may be identified via a high ventricular rate.

In at least some examples, the bradyarrhythmia condition 508 includes, but is not limited to, the heart rate being abnormally slow. In some examples, the threshold for bradyarrhythmia is defined as a heart rate of 60 beats per minute or less. The bradyarrhythmia condition 508 may be caused by conditions such as sinusbradycardia, sinoatrial block and/or atrioventricular block.

In at least some examples, chronotropic incompetence condition 509 corresponds to the inability of the heart to increase its rare commensurate with increased activity or demand, such as a steady or falling heart rate coincident with an elevated or increasing respiratory rate.

In some examples, other condition 152 includes other cardiac conditions, which may or may not be formally recognized as negative cardiac conditions or cardiac disorders but for which treatment may be desirable.

In some examples, combination condition 514 represents the existence of and/or combined effect of multiple cardiac conditions.

FIG. 15B is a block diagram schematically representing a condition determination portion 530, according to one example of the present disclosure. The condition determination portion 530 includes a heart rate parameter 532, a cardiac timing parameter 534, other parameter 536, and a combination parameter 538. In some instances, as noted in association with at least FIG. 15A, the behavior of heart rate alone may be indicative of some cardiac conditions while in some instances, heart rate information paired with other respiratory, cardiac, sleep information may be indicative of some cardiac conditions. Similarly, as noted in association with at least FIG. 15A, cardiac timing alone may be indicative of some cardiac conditions while in some instances, cardiac timing information paired with other respiratory, cardiac, sleep information may be indicative of some cardiac conditions.

It will be understood that in some examples, cardiac timing refers to observing a pattern of behavior of the operation of different portions of the heart or of behavioral aspects of the heart as the heart attempts to repeat the cardiac cycle. For instance, observing atrial-atrial timing is one form of cardiac timing that may be indicative of atrial fibrillation. Similarly, in one instance, observing a ventricular-to-atrial beat ratio is one form of cardiac timing which may be indicative of atrial fibrillation or ventricular arrhythmia, depending on the value of the ratio. In some examples, such relationships are identifiable and displayable via various tables, graphs, and/or user interfaces, as illustrated in at least some of FIGS. 4A-5B and FIGS. 13A-13I.

FIG. 15C is a block diagram schematically representing a determination engine 570, according to one example of the present disclosure. In some examples, determination engine 570 comprises at least some of substantially the same features and attributes as monitoring resource 60 as previously described in association with at least FIG. 3A. In some examples, at least some aspects of determination engine 570 may form part of monitoring resource 60 (FIG. 3A). In some examples, the determination engine 570 includes cardiac condition parameter 572, notification function 574, notification criteria 576, variances parameter 578, threshold parameter 580, responsive parameter 582, and non-responsive parameter 584.

In some examples, the determination engine 570 determines a wide variety of physiologic information regarding the patient. In some examples, this determined information may include cardiac information such as positive cardiac conditions (e.g. cardiac health conditions) and/or negative cardiac conditions (e.g. cardiac disorders), either of which are represented by cardiac condition parameter 572 in FIG. 15C. In some examples, this determined information may also include sleep quality information and/or sleep disordered breathing-related information.

In some examples, the determination engine 570 is dedicated to determining cardiac information such as positive and/or cardiac conditions. Meanwhile, in some examples, the determination engine is dedicated to tracking solely negative cardiac conditions.

In some examples, a cardiac disorder parameter represents a plurality of cardiac disorders and determination engine 570 (of the monitoring resource 60) may differentiate between a first class of the respective cardiac disorders and a second class of the respective cardiac disorders. The first class of respective cardiac disorders may correspond to a negative cardiac condition present before and after obstructive sleep apnea treatment via the system through the monitoring period. The second class of respective cardiac disorders corresponds to a presence of a negative cardiac condition present before obstructive sleep apnea treatment via the system through the monitoring period and a substantial decrease (e.g. diminishing, subsiding) in the negative cardiac condition after obstructive sleep apnea treatment via the system through the monitoring period.

Because at least some cardiac conditions are determined based on more fundamental physiologic information, such as heart rate (e.g. 532 in FIG. 15B) or cardiac timing (e.g. 534 in FIG. 15B), the information determined via determination engine 570 includes the heart rate parameter 532, cardiac timing parameter 534, and/or other physiologic information parameter 536 as shown in FIG. 15B.

The notification function 574 can deliver a notification taking the form of an notification to the user or clinician, which is communicated via text (e.g. SMS), email, audible notification, pop-up window, etc., in some form of user interface (e.g. user interface 140 in FIG. 3C) accessible by the patient and/or clinician. In some instances, such user interface may be accessed via one of the access tools 131-135 previously described in association with at least FIG. 3B, and which may include a patient programmer, clinician programmer, computer, tablet, smart phone, phablet, etc. Such devices may or may not be dedicated for use with the determination engine 570, monitoring system and/or therapy system associated with the patient.

In some examples, the notification criteria 576 provides a criteria to be met before the determination engine 570 implements a notification via notification function 574. In some examples, the notification criteria 576 is selectively adjustable by the clinician as to what conditions or information is used and/or as to which values (e.g. quantity, amplitude, frequency, duration, etc.) of a particular parameter are used to form the notification criteria 576. In some examples, the notification criteria 576 corresponds to at least some of the aspects of a diagnosis criteria used to diagnose a particular cardiac condition. In some examples, the notification criteria 576 is separate from, and independent of, such diagnosis criteria.

In some examples, notification function 574 acts to implement a notification to a patient or clinician regarding the identification of a cardiac condition. In some examples, the notification function 574 is limited to providing notifications upon the notification criteria 576 being met.

In some examples, variances function 578 determines the extent to which a particular parameter exhibits variances from expected behaviors or patterns. For instance, when determining heart rate parameter 532 (FIG. 15B), determining variances or variability in the heart rate may indicate a negative cardiac condition while a stable heart rate may indicate a positive cardiac condition or successful treatment of a negative cardiac condition or successful treatment of a sleep disordered breathing condition.

In some examples, threshold function 580 is used to set a threshold at which sensed physiologic information is deemed to correspond to a particular cardiac condition. However, in some examples, several types of physiologic information are involved in determining a cardiac condition, such that meeting a threshold for just one sensed physiologic information may not result in determination of a cardiac condition.

In some examples, the notification threshold may be automatically determined from baseline data, which is developed upon determining the threshold at which a physician typically takes action or responds to the notification. In some examples, the notification threshold is selected by the clinician in advance.

In some examples, the determination engine 570 includes a responsive parameter 582 to facilitate determination of any cardiac conditions which may be responsive (negatively or positively) to treatment of sleep disordered breathing during or after the monitoring period (e.g. 124 in FIG. 3A). In some instances, the responsive parameter 582 may facilitate a clinician in determining which, if any, cardiac conditions were alleviated as a beneficial consequence of treatment of sleep disordered breathing such that one may forego direct treatment of the cardiac condition.

In some examples, the determination engine 570 includes a non-responsive parameter 584 to facilitate determination of any cardiac condition which may be non-responsive to treatment of sleep disordered breathing during or after the monitoring period (e.g. 124 in FIG. 3A). In some instances, the non-responsive parameter 584 may facilitate a clinician in determining which, if any, cardiac conditions may be alleviated via treatment directly regarding the cardiac condition because the particular cardiac condition was not alleviated as a consequence of treatment of sleep disordered breathing. In this way, treatment of sleep disordered breathing along with the coincident monitoring of cardiac conditions may help eliminate variables in determining which therapies to which the cardiac condition may be responsive.

FIG. 16A is a block diagram schematically representing a therapy device 650, according to one example of the present disclosure. In some examples, therapy device 650 includes at least some of substantially the same features and attributes as system 50 in FIG. 1 and the various examples as previously described in association with FIGS. 1-15C.

As shown in FIG. 16A, therapy device 650 includes non-cardiac pulse generator 652, sensor 654, stimulation element 660, and a monitoring resource 664 to determine cardiac-related information 667 according to a monitoring period 668. In some examples, the cardiac-related information 667 may comprise at least the cardiac condition information 500 in FIG. 15A.

In some examples, non-cardiac pulse generator 652 comprises at least some of substantially the same features as the pulse generators previously described in association with at least FIGS. 6A, 6C, 7. In some examples, sensor 654 comprises at least some of substantially the same features as the sensor(s) 370, 400 as previously described in association with at least FIGS. 11-12.

In some examples, stimulation element 660 of device 650 comprises at least some of substantially the same features as the stimulation element(s) as previously described in association with at least FIGS. 6A and 7 such that stimulation element is operated according to a treatment period 662. In some examples, stimulation element 660 is operated to stimulate upper-airway-related body tissue (e.g. 180 in FIG. 6B) to restore upper airway patency.

In some examples, device 650 monitors a cardiac condition 664 according to a monitoring period 668, in a manner at least consistent with the monitoring of cardiac conditions, as previously described in association with at least FIGS. 1-15C.

In some examples, device 650 may further include a therapy manager (e.g. 110 in FIG. 3A) and/or control portion 880 having manager 885 (FIG. 23). In such examples, the manager and/or control portion may coordinate stimulation via the stimulation element 660 according to the treatment period 662 (also 112 in FIG. 3A) and/or may monitor cardiac conditions 670 according to the monitoring period 668 (also 124 in FIG. 3A).

FIG. 16B is a diagram schematically representing a stimulation system 670, according to an example of the present disclosure. As illustrated in FIG. 16B, in some examples system 670 comprises an implantable pulse generator (IPG) 675, capable of being surgically positioned within a pectoral region of a patient 671, and a stimulation lead 674 electrically coupled with the IPG 675. In some examples, pulse generator 675 comprises at least some of substantially the same features and attributes as the pulse generator 200, as previously described in association with at least FIG. 6A, 6C, 7 and the various examples described throughout the present disclosure.

The lead 672 includes a stimulation element 676 (e.g. electrode portion, such a cuff electrode) and extends from the IPG 675 so that the stimulation element 690 is positioned in contact with a desired nerve 673 to stimulate nerve 673 for restoring upper airway patency. In some examples, the desired nerve comprises a hypoglossal nerve. In some examples, stimulation element 676 comprises at least some of substantially the same features and attributes as the stimulation element 174, 216, as previously described in association with at least FIGS. 6A and 7, and the various examples described throughout the present disclosure. In some instances, a body of the stimulation lead 674 may sometimes be referred to as being interposed between, and extending between the IPG 675 and the stimulation element 676.

One implantable stimulation system in which lead 672 may be utilized, for example, is described in U.S. Pat. No. 6,572,543 to Christopherson et al., and which is incorporated herein by reference in its entirety. In one example, device 670 comprises includes at least one sensor portion 680 (electrically coupled to the IPG 675 and extending from the IPG 675 via lead 677) positioned in the patient 671 for sensing respiratory effort, such as respiratory pressure.

In some examples, the sensor portion 680 detects respiratory effort including respiratory patterns (e.g., inspiration, expiration, respiratory pause, etc.). In some examples, this respiratory information is employed to trigger activation of stimulation element 676 to stimulate a target nerve 673. Accordingly, in some examples, the IPG 675 receives sensor waveforms (e.g. one form of respiratory information) from the respiratory sensor portion 680, thereby enabling the IPG 675 to deliver electrical stimulation according to a therapeutic treatment regimen in accordance with examples of the present disclosure. In some examples, the respiratory information is used to apply the stimulation synchronously with inspiration or synchronized relative to another aspect of the respiratory cycle. In some examples, this arrangement may sometimes be referred to as closed-loop stimulation. In some examples, the respiratory sensor portion 680 is powered by the IPG 675.

In some examples, stimulation may be applied without synchronization relative to a portion of the respiratory cycle, and therefore may sometimes be referred to as open-loop stimulation or therapy.

In some examples, sensor portion 680 comprises at least some of substantially the same features and attributes as sensor(s) 370 and 400, as previously described in association with at least FIGS. 11-12 and the various examples described throughout the present disclosure.

Accordingly, in some examples, the sensor portion 680 comprises a pressure sensor, such as pressure sensor 402 (FIG. 12). In one example, the pressure sensor in this example detects pressure in the thorax of the patient. In other examples, the sensed pressure can be a combination of thoracic pressure and cardiac pressure (e.g., blood flow). With this configuration, a controller associated with IPG 675 is configured to analyze this pressure sensing information to detect the respiratory patterns of the patient.

In some other examples, the respiratory sensor portion comprises a bio-impedance sensor or an array of bio-impedance sensors and can be located in regions other than the pectoral region. In one aspect, such an impedance sensor is configured to sense a bio-impedance signal or pattern whereby the control unit evaluates respiratory patterns within the bio-impedance signal. For bio-impedance sensing, in one example, electric current will be injected through an electrode portion within the body and an electrically conductive portion of a housing (i.e. case, can, etc.) of the IPG 675 with the voltage being sensed between two spaced apart stimulation electrode portions (such as stimulation element 676), or also between one of the stimulation electrode portions and the electrically conductive portion of the case of IPG 675 to compute the impedance.

In some examples, system 670 comprises other sensors (instead of sensor portion 680) or additional sensors (in addition to sensor portion 680) to obtain physiologic data associated with respiratory functions. For instance, as shown in FIG. 16B, in some examples system 670 may include various electrode portions 682, 683, 684 distributed about the chest area for measuring a trans-thoracic bio-impedance signal, an electrocardiogram (ECG) signal, or other respiratory-associated signals, other cardiac signals, etc.

In some examples, the various electrode portions 682, 683, 684 or even a single lead is used to measure trans-thoracic electrical bio-impedance in combination with obtaining a far field ECG to filter/blank cardiac artifacts from the bio-impedance signal. In some examples, the trans-thoracic bio-impedance signal may be used to determine cardiac output and respiratory output (e.g. minute ventilation). For instance, the thoracic bio-impedance may provide a relative measure of respiratory output and stroke volume, and thereby provide a custom ventilation parameter, which in turn may be used in a self-developing correlation vector (as later described in association with at least FIG. 22) to monitor changes over time (such as during the monitoring period 124 in FIG. 3A).

In some examples, the sensing and stimulation system for treating sleep disordered breathing (such as but not limited to obstructive sleep apnea) is a totally implantable system which provides therapeutic solutions for patients diagnosed with obstructive sleep apnea. In other examples, one or more components of the system are not implanted in a body of the patient, as was previously noted for the examples of external components 204 of non-cardiac pulse generator 200 in association with FIG. 6C. A few non-limiting examples of such non-implanted components include external sensors (respiration, impedance, etc.), an external processing unit, or an external power source. Of course, it is further understood that, in some examples, the implanted portion(s) of the system provides a communication pathway to enable transmission of data and/or controls signals both to and from the implanted portions of the system relative to the external portions of the system. The communication pathway includes a radiofrequency (RF) telemetry link or other wireless communication protocols.

Whether partially implantable or totally implantable, the system is designed to stimulate an upper-airway-patency-related nerve during some portion of the repeating respiratory cycle to thereby prevent obstructions or occlusions in the upper airway during sleep.

In some examples, among other potential functions, the pulse generator 675 includes a sensing engine 690, stimulation engine 692, and a therapy manager 694 and control portion 696, as shown in FIG. 16C. In some examples, the control portion 696 comprises at least some of substantially the same features and attributes as control portion 880 as described in association with FIG. 23.

In some examples, the pulse generator 675 includes a monitoring resource having at least some of substantially the same features and attributes as monitoring resource 60 as previously described in association with at least FIGS. 1, 3A and other monitoring resources described throughout the examples of the present disclosure.

Via an array of parameters, the sensing engine 690 receives and determines signals from various physiologic sensors (such as a pressure sensor, blood oxygenation sensor, acoustic sensor, electrocardiogram (ECG) sensor, or impedance sensor as described in association with at least FIGS. 11-12) in order to determine a respiratory state of a patient, whether or not the patient is asleep or awake, and other respiratory-associated indicators, etc. Such respiratory detection may be received from either a single sensor or any multiple of sensors, or combination of various physiologic sensors which may provide a more reliable and accurate signal. In one example, sensing engine 690 receives signals from sensor portion 680 and/or sensors 682, 683, 684 in FIG. 16B, or any of the sensor(s) as previously described in association with at least FIGS. 11-12.

In some examples, sensing engine 690 cooperates with, is in communication with, and/or forms part of a monitoring resource (e.g. at least 60 in FIG. 3A; 570 in FIG. 15) to receive, determine, and/or monitor at least the parameters, information and conditions, as previously described in association with at least FIGS. 1-15C.

In some examples, among other functions the therapy manager 694 of pulse generator 675 acts to synthesize respiratory information, to determine suitable stimulation parameters (via stimulation engine 692) based on that respiratory information, and to direct electrical stimulation to the target nerve. In some examples, therapy manager 694 may comprise at least some of substantially the same features and attributes of control portion 880 and/or may cooperate with control portion 880 in FIG. 23.

FIG. 17A is a block diagram schematically representing a monitoring resource 700, according to one example of the present disclosure. As shown in FIG. 17A, monitoring resource 700 includes sensor 702 and monitoring engine 704.

In some examples, sensor 702 includes at least some of substantially the same features as the sensors previously described in association with at least FIGS. 11-12 and with FIGS. 13A-15C. Accordingly, the sensor(s) 702 may be internal (e.g. implanted within the patient) or external to the patient, or a combination of both internal and external. When external, the sensors may be wearable by the patient, removably securable to the patient, or part of the patient's environment.

In some examples, the monitoring engine 704 monitors sleep parameter 706 and cardiac parameter 708 regarding the patient. In some examples, the cardiac parameter 708 includes at least some of substantially the same features and attributes as cardiac parameters (62 in FIG. 1, 3A; 64 in FIG. 2A; 66 in FIG. 2B; 304 in FIG. 9) and cardiac information (FIGS. 13-15) as disclosed throughout the present disclosure.

In some examples, monitoring resource 700 is separate from, and independent of, a therapy device but may communicate with a therapy device, such as (but not limited to) one of the therapy devices described in at least some examples of the present disclosure. In some examples, monitoring resource 700 forms part of, or cooperates with, a therapy device, such as one of the therapy devices described in at least some examples of the present disclosure.

In some examples, monitoring resource 700 forms part of and/or cooperates with a therapy manager (694 in FIG. 16C) while in some examples, monitoring resource 700 is separate from, and independent of, such managers but may communicate with such managers.

In some examples, monitoring resource 700 is implemented within and/or forms a standalone device. In some examples, monitoring resource 700 is incorporated within and forms an application on a mobile device (e.g. 131, 132 in FIG. 3B). In some instances, the mobile device (e.g. 131 in FIG. 3B) is dedicated to monitoring cardiac parameters and/or sleep disordered breathing parameters while in some instances, the mobile device (e.g. 132 in FIG. 3B) is a non-dedicated mobile device, such as but not limited to, a smart phone, tablet, phablet, notebook computer, etc.

FIG. 17B is a block diagram schematically representing a monitoring resource 710, according to one example of the present disclosure. As shown in FIG. 17B, monitoring resource 710 comprises at least some of substantially same features and attributes as monitoring resource 700 in FIG. 17A, except with sensor 712 and/or information 714 being separate from and/or independent of monitoring resource 710, and with monitor resource 710 being in cooperation with and/or in communication with sensor 712 and/or information 714.

FIG. 18A is a block diagram schematically representing a manager 750, according to one example of the present disclosure. As shown in FIG. 18A, monitoring resource 750 comprises a monitoring engine 752 and an evaluation engine 758. The determination engine 752 monitors at least an array of sleep parameters 754 and an array of cardiac parameters 756. The evaluation engine 758 evaluates the monitored parameters 754, 756 looking for positive and negative correlations of the parameters relative to each other, as will be further described later in association with at least FIG. 22. In some examples, the sleep parameters 754 comprise sleep quality parameters, sleep disordered breathing parameters, among other sleep-related parameters. In some examples, the sleep disordered breathing parameters comprise at least obstructive sleep apnea-related parameters. In some examples, the obstructive sleep apnea-related parameters comprise various physiologic parameters associated with the presence or absence of obstructive sleep apnea.

In some examples, some of the cardiac parameters may comprise a cardiac disorder parameter. In some examples, the cardiac disorder parameters 756 are associated with negative cardiac conditions. However, in some examples, a cardiac disorder parameter 756 may be associated with a positive cardiac condition. In some examples, the cardiac conditions comprise various physiologic parameters associated with the presence or absence of cardiac conditions.

FIG. 18B is table identifying at least some sleep/sleep quality parameters, at least some cardiac conditions/parameters, and other parameters, according to one example of the present disclosure. Any one of the sleep/sleep quality parameters, the cardiac condition/parameters, the other parameters, and/or the pulmonary parameters can be correlated with each other as driven by the actual physiologic behavior of the patient.

It will be understood that in some examples, via analytic tools, the various sleep quality parameters and cardiac parameters may be organized manually or automatically (via self-development) into other formats, matrices, grids, and/or multi-dimensional forms, which reflect the functional or correlational relationship among the respective sleep and cardiac parameters. At least some examples are provided throughout the Figures, including but not limited to, at least FIGS. 4A-5B and 13A-13I.

In some examples, each sleep/sleep quality parameter is compared relative to a first criteria for that respective sleep/sleep quality parameter, and in some examples, each cardiac condition/parameter is compared relative to a second criteria for that particular cardiac condition/parameter.

In some examples, via evaluation engine 758 monitoring resource 750 (FIG. 18A) automatically determines uniquely for each patient any positive sleep quality parameters characterized by their improvement with SDB treatment associated with the monitoring period and any negative sleep quality parameters characterized by their deterioration with SDB treatment associated with the monitoring period.

In some examples, via evaluation engine 758 (of monitoring resource 750) automatically determines uniquely for each patient any cardiac disorder parameters characterized by their decrease with SDB treatment associated with the monitoring period and any cardiac disorder parameters characterized by their persistence despite SDB treatment associated with the monitoring period.

In some examples, the evaluation engine 758 (of monitoring resource 750) determines a correlation of positive sleep quality parameters and decreased cardiac disorder parameters. In some examples, the evaluation engine 758 determines a correlation of negative sleep quality parameters and persistent cardiac disorder parameters.

In some examples, the evaluation engine 758 (of monitoring resource 750) determines a correlation of positive sleep quality parameters and persistent cardiac disorder parameters. In some examples, the evaluation engine determines a correlation of negative sleep quality parameters and improved cardiac disorder parameters.

In some examples, the first criteria set includes a separate criteria/threshold for each different sleep quality parameter and the second criteria/set includes a separate criteria/threshold for each different cardiac disorder parameter.

In some examples, one sleep quality parameter includes determining a duration and quantity of non-REM and REM sleep stages, as well as a total duration of sleep. In some examples, an accelerometer (e.g. accelerometer 406 in FIG. 12) is used to determine a duration of sleep and/or soundness of sleep via sensing body motion, body activity, body position(s), and/or body posture.

In some examples, at least some patient data which was determined during or after a monitoring period can be displayed via a graph 760 such as shown in FIG. 18C, according to one example of the present disclosure. As shown in FIG. 18C, in some examples graph 760 displays information about at least one sleep disordered breathing (SDB) parameter 761A, at least one cardiac parameter 761B, and at least one other parameter 761C (e.g. pulmonary). In one instance, the at least one SDB parameter 761A comprises an apnea-hypopnea index (AHI) while the at least one cardiac parameter 761B comprises an average heart rate variability (HRV). It will be understood that in at least some examples, the apnea-hypopnea index (AHI) may correspond to a quantity of apneas over a time period. In one instance, the other parameter 761C comprises a respiratory rate. It will be understood that many other parameters from each of the respective categories of sleep disordered breathing, cardiac, and other/pulmonary can be selected for display and comparison on graph 760 instead of or in addition to those shown in the example of FIG. 18C.

In some examples, graph 760 displays the respective parameters 761A, 761B, 761C as box-and-whisker plots as shown in FIG. 18C in which a box (e.g. 762A, 762B, 762C) graphically demonstrates a range of primary values for each respective parameter 761A-761C and the whiskers (e.g. 763A, 763B, 763C) extending from each end of the respective box identify a number of data points outside the primary range. By aligning the box-and-whisker plots for the parameters relative to each other, one can then correlate when both the SDB parameter 761A and the cardiac parameter 761B are both out of the primary range (e.g. box) at the same time. Stated differently, one can observe where the respective whiskers overlap. Moreover, once any such correlation is identified, further filtering can be applied to other parameters (such as those at least some of the parameters listed in Table of FIG. 18B) to observe behavior of other parameters during those times and potentially identify further correlation(s) between those “other” parameters and the SDB parameter 761A and cardiac parameter 761B. It will be understood in some examples, the sleep quality parameter 902 of an apnea-hypopnea index (AHI) (e.g. number of apnea events per unit of time) may be substituted by a total quantity of obstructive sleep apnea events, a severity of apnea event(s), etc. In some examples, the patient data shown in graph 760 of FIG. 18C is obtained during or after a monitoring period (e.g. 124 in FIG. 3A). In some instances, the monitoring period may be relatively long term, such as but not limited to, one year such as might occur upon a patient having a one year check-up with a clinician. In such instances, the values represented in the box-and-whisker plots would correspond to one year of data, such that any correlations derivable from the plots may demonstrate long term trends regarding a patient's cardiac conditions (positive or negative) over that time period relative to the patient's SDB therapy over that same time period.

With further reference to FIG. 18A, in some examples monitoring resource 750 comprises a portion of a therapy device 765, as shown in FIG. 19, in which therapy device 765 includes non-cardiac stimulator circuitry 767, which can take the forms previously described in association with at least FIGS. 6A, 6C, 7, 10A-10B, and 16A-16B, or other forms The non-cardiac stimulator circuitry 767 is configurable to stimulate upper-airway-patency related body tissues, such as nerves, muscles, etc.

In some examples, the non-cardiac stimulator circuitry 767 may comprise a transvenously implantable stimulation element operably couplable relative to an external pulse generator. In some examples, such non-cardiac stimulator circuitry may comprise a percutaneously implantable stimulation element wirelessly operably couplable relative to an external pulse generator. In either case, when coupled together in this manner, power, data, and/or control may be transferred wirelessly between the implantable stimulation element and the external pulse generator.

In either the transvenous or percutaneous modality, in some such examples, some components associated with pulse generation and/or control may be implantable in proximity to or co-located with the implantable stimulation element.

In some examples, monitoring resource 750 is separate from, and independent of, a therapy device (e.g. 765 in FIG. 19) but may communicate with or cooperate with such therapy devices.

In some examples, therapy device 765 includes a wireless communication link 768 (FIG. 20) for receiving and/or obtaining the information (e.g. 300 in FIG. 9 and FIGS. 13A-14B) for determining via determination engine 752 (FIG. 18A).

In some examples, therapy device 765 includes or is in communication with a sensor 769 (FIG. 21) for receiving and/or obtaining the information (e.g. 300 in FIG. 9 and FIGS. 13A-14B) for determining via determination engine 752 (FIG. 18A). In some examples, sensor 769 comprises at least some of substantially the same features and attributes as the sensor(s) as previously described in association with at least FIGS. 11-12.

FIG. 22 is block diagram schematically representing an evaluation engine 770, according to one example of the present disclosure. In some examples, evaluation engine 770 serves as evaluation engine 758 in the examples of FIGS. 18A-19. As shown in FIG. 20, in some examples evaluation engine 758 comprises a correlation function 772, correlation criteria 790, notification criteria 792, and patient compliance parameter 794.

Correlation function 772 operates to identify and determine correlations among different determined parameters, such as but not limited to, sleep quality parameters 754 and cardiac disorder parameters 756 as provided in determination engine 752 of FIG. 18A. In some examples, pulmonary parameters and/or other parameters are determined and correlated along with the sleep quality and cardiac parameters 754, 756. In some examples, correlation function 772 operates via an automatic mode 774 in which such correlations are automatically determined via statistical analysis and/or predetermined correlation metrics, such as correlation criteria 790. In some examples, correlation function 772 operates via a manual mode 776 in which such correlations are identified manually.

Notification criteria 792 enables setting a criteria which is to be met before a notification (e.g. 574 in FIG. 15C) is made to a clinician regarding any identified correlation. In some examples, notification criteria 792 comprises at least some of substantially the same features and attributes as notification criteria 576 as previously described in association with at least FIG. 15C.

Patient compliance parameter 794 enables determining the extent to which a patient has been compliant with a therapy for treating sleep disordered breathing, thereby equipping a clinician or evaluator to weigh this patient compliance as a factor when evaluating any notification regarding a correlation identified via correlation function 772. In some instances, the patient compliance parameter 794 may be expressed as a usage parameter, which may form part of a self-developing correlation vector regarding combinations of positive parameters (i.e. those contributing to efficacious therapy) or a self-developing correlation vector regarding combinations of negative parameters (i.e. those contributing to a lack of efficacious therapy).

In some examples, evaluation engine 770 includes an array 779 of evaluative operators, such as but not limited to, positive parameter 780, negative parameter 781, increase parameter 782, decrease parameter 783, persistence parameter 784, subside parameter 785, and threshold parameter 786 for identifying associated values of determined parameters (754, 756 in FIG. 18A) and/or identifying associated correlations among the determined parameters. In some instances, an increase in a positive parameter sometimes may be referred to as an improvement while in other instances, a decrease (or subsiding) in a negative parameter sometimes may be referred to as an improvement.

In some examples, during or after a monitoring period, the evaluation engine 770 may automatically identify associations and/or correlations between sleep quality parameters 754 and cardiac disorder parameters 756 (FIG. 18A). In this way, the evaluation engine 770 enables automatic development of a correlation vector for a particular patient during or after a monitoring period, wherein the correlation vector reflects some relationship among sleep quality parameters 754 and cardiac disorder parameters 756 such that treatment of sleep disordered breathing may result in a decrease (e.g. 783 in FIG. 22) in, or subsiding (e.g. 785 in FIG. 22) of, an existing cardiac disorder or may result in the persistence (e.g. 784 in FIG. 22) of a cardiac disorder despite treatment of sleep disordered breathing. In some instances, a decrease or subsiding for a positive parameter may be referred to a deterioration. In some instances, in an increase in a negative parameter sometimes may be referred to as a deterioration.

For instance, in some examples, during or after a monitoring period, the evaluation engine 770 may identify that a cardiac condition such as atrial fibrillation persists despite treatment for sleep disordered breathing. Upon confirmation that the sleep disordered breathing treatment was effective, it may then be determined that the atrial fibrillation may have causes unrelated to the sleep disordered breathing previously exhibited by the patient. A clinician may then recommend other therapeutic steps to alleviate the cardiac disorder (e.g. atrial fibrillation), such as drug therapy, surgery, ablation, electrically stimulating a portion of the heart (e.g. pacing, defibrillation, etc.), and/or non-hypoglossal nerve stimulation such as stimulating the vagus nerve.

Alternatively, during or after a monitoring period, the evaluation engine 770 may identify that a cardiac condition, such as atrial fibrillation subsides or decreases during or after treatment for sleep disordered breathing. Upon confirmation that the sleep disordered breathing treatment was effective, it may then be determined that the sleep disordered breathing previously exhibited by the patient was at least partially responsible for the previously exhibited atrial fibrillation.

In some examples, a correlation between a patient compliance/usage parameter 794 of the SDB therapy device and cardiac parameters may allow for a single variable to indicate the efficacy of the SDB treatment. A low number could signal that re-programming of the SDB therapy device is recommended to improve SDB therapy efficacy or that referral to a cardiac health specialist is appropriate. In this way, leading indicators to treat cardiac health (in the specific context of SDB therapy) may help the long term health of patients.

In some examples, one correlation vector comprises an atrial fibrillation burden parameter vs. patient compliance for SDB therapy vs. SDB efficacy. This correlation vector may be useful to notify clinicians in taking early action regarding either the SDB therapy and/or the atrial fibrillation behavior. For instance, if the atrial fibrillation burden persists (e.g. persistence parameter 784 in FIG. 22) even with a high value of SDB efficacy and a high value of SDB therapy patient compliance, this correlation may be an indication of a structural cardiac issue and that the patient may benefit from an interventional cardiac procedure such as, but not limited to, ablation to treat the atrial fibrillation behavior. In this way, the correlation vector helps to identify cardiac parameters that are not positively responsive to SDB therapy.

In some examples, the atrial fibrillation burden can be quantified in at least two ways. For instance, the atrial fibrillation burden can be quantified via RR interval variability (where R refers to the R in a QRS complex of a cardiac waveform) or via atrial-atrial (AA) timing vs ventricle-ventricle (VV) timing.

It will be understood that the self-developing correlation vector of sleep quality parameters 754 and cardiac disorder parameters 756 may develop associations and/or correlations between respective parameters 754 and 756 which are unique for a particular patient and not necessarily exhibited by a larger patient population as a whole. This arrangement may lead to unique treatment options for a particular patient. Moreover, in some instances, any correlation data which is self-developed for each patient may be aggregated with self-developed correlation data from other patients to enable determining correlations (or a lack of correlation) among at least some sleep quality parameters 754 (which includes, but it is not limited to, sleep disordered breathing parameters) and at least some cardiac disorder parameters 756 which are common among a group of patients.

FIG. 23 is a block diagram schematically representing a control portion 880, according to one example of the present disclosure. In some examples, control portion 880 includes a controller 882 and a memory 884. In some examples, control portion 880 provides one example implementation of a control portion forming a part of, or implementing, any one of managers, monitoring resource, determination engines, and/or therapy devices/systems, as represented throughout the present disclosure in association with FIGS. 1-22.

In general terms, controller 882 of control portion 880 comprises at least one processor 883 and associated memories. The controller 882 is electrically couplable to, and in communication with, memory 884 to generate control signals to direct operation of at least some components of the systems, devices, components, monitoring resource, managers, functions, parameters, and/or engines described throughout the present disclosure. In some examples, these generated control signals include, but are not limited to, employing engine 885 stored in memory 884 to manage therapy for a patient, provide sleep monitoring, and/or provide cardiac monitoring, in the manner described in at least some examples of the present disclosure. It will be further understood that control portion 880 (or another control portion) may also be employed to operate general functions of the various therapy devices/systems, access tools 131-135 (FIG. 3B) described throughout the present disclosure.

In response to or based upon commands received via a user interface (e.g. user interface 140 in FIG. 3C) and/or via machine readable instructions, controller 882 generates control signals to implement therapy implementation, monitoring, management, sleep monitoring, and/or cardiac monitoring in accordance with at least some of the previously described examples of the present disclosure. In some examples, controller 882 is embodied in a general purpose computing device while in other examples, controller 882 is embodied in a monitoring resource generally or incorporated into or associated with at least some of the related components described throughout the present disclosure.

For purposes of this application, in reference to the controller 882, the term “processor” shall mean a presently developed or future developed processor (or processing resource(s)) that executes sequences of machine readable instructions contained in a memory. In some examples, execution of the sequences of machine readable instructions, such as those provided via memory 884 of control portion 880 cause the processor to perform actions, such as operating controller 882 to implement therapy, sleep monitoring, and/or cardiac monitoring, as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 884. In some examples, memory 884 comprises a computer readable tangible medium providing non-volatile storage of the machine readable instructions executable by a process of controller 882. In other examples, hard wired circuitry may be used in place of or in combination with machine readable instructions to implement the functions described. For example, controller 882 may be embodied as part of at least one application-specific integrated circuit (ASIC). In at least some examples, the controller 882 is not limited to any specific combination of hardware circuitry and machine readable instructions, nor limited to any particular source for the machine readable instructions executed by the controller 482.

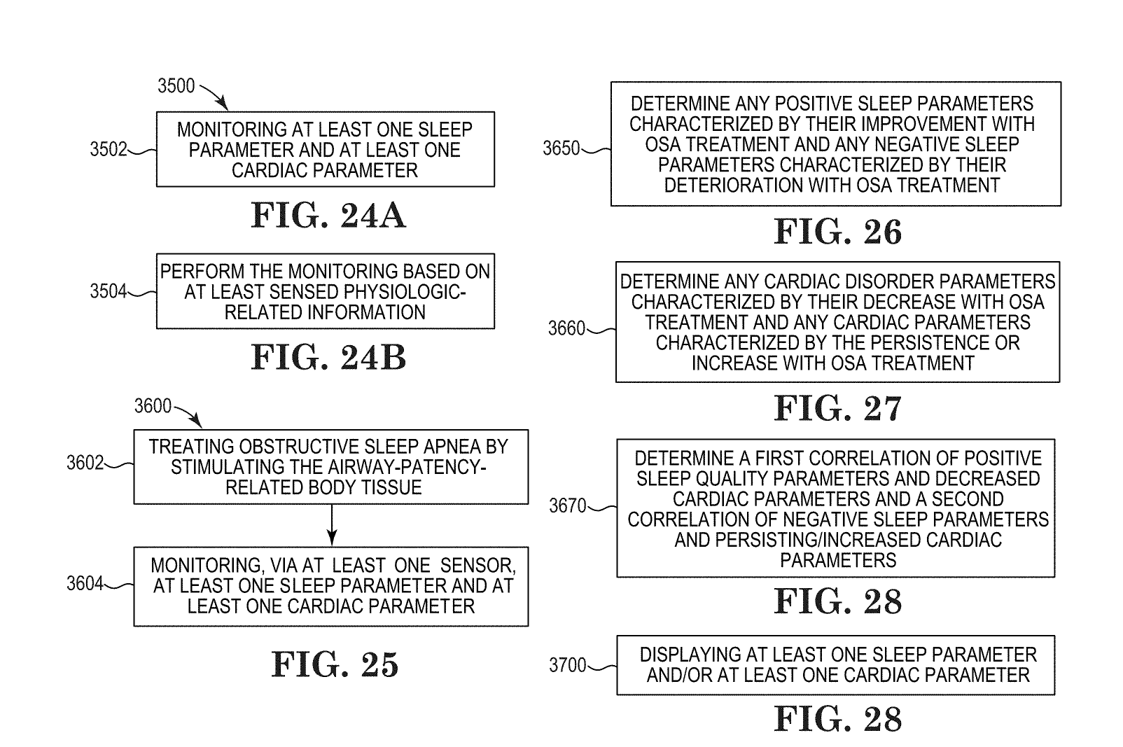

FIG. 24A is a block diagram schematically representing instructions 3502, according to one example of the present disclosure.

With regard to the instructions 3502 (FIG. 24A), 3504 (FIG. 24B) and the instructions 3600 (FIG. 25), 3650 (FIG. 26), 3660 (FIG. 27), 3670 (FIG. 28), 3700 (FIG. 29), in some examples, any or all of the instructions 3500, 3600, 3650, 3660, 3670, 3700 may be implemented via at least some of substantially the same systems, devices, functions, parameters, engines, monitoring resource, modules, managers, elements, components, instructions, etc. as previously described in association with at least FIGS. 1-23. In some examples, any or all of the respective instructions may be implemented via at least some systems, devices, functions, parameters, engines, monitoring resource, modules, managers, elements, components, instructions, etc. other than those previously described in association with at least FIGS. 1-23. Moreover, the respective instructions represented in association with at least FIGS. 24-29 may be combined with other instructions associated with the various systems, devices, functions, parameters, engines, monitoring resource, modules, managers, elements, components, etc. as previously described in association with at least FIGS. 1-23.

In addition, regarding the instructions 3502 (FIG. 24A), 3504 (FIG. 24B) and the instructions 3600 (FIG. 25), 3650 (FIG. 26), 3660 (FIG. 27), 3670 (FIG. 28), 3700 (FIG. 29), in some examples, any or all of the instructions 3502, 3504, 3600, 3650, 3660, 3670, 3700 may be implemented as a method via at least some of substantially the same systems, devices, functions, parameters, engines, monitoring resource, modules, managers, elements, components, instructions, features, attributes, etc. as previously described in association with at least FIGS. 1-23.

As shown in FIG. 24A, at 3502 the instructions 3500 comprise monitoring at least one sleep parameter and at least one cardiac parameter.

FIG. 24B is a block diagram schematically representing instructions 3504, according to one example of the present disclosure. As shown in FIG. 24B, at 3504, the instructions comprise performing the monitoring based on at least sensed physiologic-related information. In some examples, instructions 3504 are implemented to complement instructions 3502.

FIG. 25 is a flow diagram schematically representing instructions 3600, according to one example of the present disclosure. As shown in FIG. 25, at 3602 the instructions 3600 comprise directing treatment of obstructive sleep apnea by stimulating the airway-patency-related body tissue via stimulator circuitry. At 3604, the instructions 3600 comprise monitoring, via at least one sensor, at least one sleep parameter and at least one cardiac parameter.

FIG. 26 is a block diagram schematically representing instructions 3650, according to one example of the present disclosure. Instructions 3650 comprise determining any positive sleep parameters characterized by their improvement with OSA treatment and any negative sleep parameters characterized by their deterioration with OSA treatment.

FIG. 27 is a block diagram schematically representing instructions 3660, according to one example of the present disclosure. Instructions 3660 comprise determining cardiac parameters characterized by their decrease with OSA treatment and any cardiac parameters characterized by their persistence and/or increase despite OSA treatment.

FIG. 28 is a block diagram schematically representing instructions 3670, according to one example of the present disclosure. Instructions 3670 comprise determining a first correlation of positive sleep parameters and decreased cardiac parameters and a second correlation of negative sleep parameters relative to persistent and/or increased cardiac disorder parameters.

In some examples, instructions 3650 (FIG. 26), instructions 3660 (FIG. 27), and instructions 3670 (FIG. 28) are implemented together in a complementary manner.

FIG. 29 is a block diagram schematically representing instructions 3700, according to one example of the present disclosure. Instructions 3700 comprise displaying at least one sleep parameter and/or at least one cardiac parameter. In some examples, instructions 3700 (FIG. 29) are implemented in a complementary manner with the instructions 3502 (FIG. 24A), instructions 3504 (FIG. 24B), instructions 3600 (FIG. 25), instructions 3650 (FIG. 26), instructions 3660 (FIG. 27), and/or instructions 3670 (FIG. 28), whether in the form of a method or otherwise as noted above.

Although specific examples have been illustrated and described herein, it will be appreciated by those of ordinary skill in the art that a variety of alternate and/or equivalent implementations may be substituted for the specific examples shown and described without departing from the scope of the present disclosure. This application is intended to cover any adaptations or variations of the specific examples discussed herein.

1. An apparatus comprising:
a monitoring resource to monitor at least one sleep-related parameter and at least one cardiac-related parameter.

2. The apparatus of claim 1, the monitoring resource to perform the monitoring at least partially based on sensed physiologic-related information.

3. The apparatus of claim 2, wherein the sensed physiologic-related information comprises sensed cardiac information, and
the monitoring resource to determine whether the at least one sensed cardiac parameter corresponds to a negative cardiac condition for the patient.

4. The apparatus of claim 3, wherein upon determination of the presence of a negative cardiac condition, the monitor resource is to produce a notification.

5. The apparatus of claim 3, wherein the negative cardiac condition comprises at least one of:
premature beats;
a supraventricular arrhythmia;
a ventricular arrhythmia;
a bradyarrhythmia;
hypertension;
heart failure; and
chronotropic incompetence.

6. The apparatus of claim 3, the monitoring resource to determine any negative cardiac conditions which are not responsive to obstructive sleep apnea treatment for the patient.

7. The apparatus of claim 3, the monitoring resource to determine any negative cardiac conditions which are responsive to obstructive sleep apnea treatment for the patient.

8. The apparatus of claim 1, the monitoring resource to determine a relationship between the respective determined at least one sleep-related parameter and the at least one cardiac-related parameter.

9. The apparatus of claim 1, the monitoring resource comprising a user interface to display a trend of the at least one cardiac parameter and the at least one sleep parameter over a monitoring period.

10. The apparatus of claim 2, the monitoring resource to receive the sensed physiologic information from an implantable sensor.

11. The apparatus of claim 1, the monitoring resource to receive the sensed physiologic information from an external sensor.

12. The apparatus of claim 12, wherein the external sensor comprises a non-contact sensor.

13. The apparatus of claim 1, comprising at least one sensor to sense the physiologic information, the at least one sensor comprises at least one of:
a pressure sensor;
an accelerometer;
an impedance sensor;
an ultrasonic sensor;
a radiofrequency sensor;
a non-contact sensor;
an optical sensor;
an acoustic sensor;
an airflow sensor;
an image sensor;
an EMG sensor; and
an ECG sensor.

14. The apparatus of claim 1, comprising:
a stimulation element to stimulate airway-patency-related body tissue according to an obstructive sleep apnea (OSA) treatment period, and the monitoring resource to perform the monitoring regarding the respective at least one sleep parameter and cardiac parameter relative to the OSA treatment period.

15. The apparatus of claim 14, comprising:
a non-cardiac pulse generator to implement the OSA treatment period via the stimulation element.

16. The apparatus of claim 1, the monitoring resource comprising:
a processing resource to execute machine readable instructions, stored in a non-transitory medium, to perform the monitoring of the at least one sleep-related parameter and the at least one cardiac-related parameter.

17. The apparatus of claim 1, wherein the monitoring resource is implemented via at least one of:
a mobile device;
a dedicated station;
a portal;
a user interface displayable via at least one of a mobile device, a dedicated station, a portal.

18. The apparatus of claim 1, wherein the monitoring resource is located at least partially external to the patient.

19. The apparatus of claim 1, the monitoring resource to perform the monitoring relative to a monitoring period.

20. The apparatus of claim 19, wherein the monitoring period is independent of an OSA treatment period, and the monitoring period has a duration at least one order of magnitude greater than a duration of the OSA treatment period.

21-115. (canceled)
